(19) United States
(12) Patent Application Publication
(10) Pub. No.: US 2026/0367029 A1
(43) Pub. Date: Nov. 19, 2026

(54) ELECTROCHEMICAL METHOD FOR DETECTING CANCER

(71) Applicants: B.G. NEGEV TECHNOLOGIES AND APPLICATIONS LTD., AT BEN-GURION UNIVERSITY, Beer Sheva (IL); SHEBA IMPACT LTD., Ramat-Gan (IL)

(72) Inventors: Hadar Shmuel BEN-YOAV, Ramat Gan (IL); Avia LAVON, Beer-Sheva (IL); Anat Tzipora TZUR, Petach Tikvah (IL); Gal MARKEL, Ramat Gan (IL); Assaf BAR EL, Ramat HaSharon (IL)

(73) Assignees: B.G. NEGEV TECHNOLOGIES AND APPLICATIONS LTD., AT BEN-GURION UNIVERSITY, Beer Sheva (IL); SHEBA IMPACT LTD., Ramat-Gan (IL)

(21) Appl. No.: 19/117,768

(22) PCT Filed: Oct. 3, 2023
(86) PCT No.: PCT/IL2023/051060

§ 371 Date: Apr. 2, 2025

Related U.S. Application Data
(60) Provisional application No. 63/412,817, filed on Oct. 3, 2022.

(51) Int. Cl. G01N 27/327

(52) U.S. Cl. CPC G01N 27/3277

(57) ABSTRACT

A method of screening and/or monitoring a patient for cancer that is detectable in urine, comprising: obtaining a urine sample from the patient; acquiring an electrochemical signal generated jointly by redox molecules in the urine sample, wherein the signal is acquired with the aid of an array comprising surface-modified electrodes that is sensitive for antioxidant and oxidant, wherein the array optionally further comprises one or more bare electrodes; optionally preprocessing the electrochemical signal, to obtain processed data; applying trained chemometric model(s) to the processed or raw data; and determining the presence of cancer and/or monitorable cancer-related features, after the chemometric model has classified the processed data according to its training data set.

### BACKGROUND OF THE INVENTION

Non-invasive methods based on the testing of biofluids to determine, for a cancer patient or a subject suspected of having cancer, which is the organ of cancer involvement, are of great importance. For example, detection of urologic cancer by the testing of urine sample. The major types of urologic cancers include prostate cancer (PC) and bladder cancer (BC), which are the second and fourth most common cancer in men, respectively. There exists a need for urine-based screening of urologic cancers, that is, by testing urine sample taken from a patient for the presence of a biomarker or a fingerprint associated with the organ of cancer involvement, e.g., the urine bladder or prostate.

Electrochemistry could be incorporated into urinalysis, to serve the purpose of cancer detection. Integration of electrochemical tools into the diagnosis of cancer is based on the inherent redox nature of most of the metabolites involved. An electrochemical approach offers reduced diagnostic time and potential miniaturization—an important consideration because a biofluid sample taken from a patient often has limited volume. Consequently, an electrochemical measurement cell should be designed to accommodate and test a small volume of the biofluid sample.

Hitherto, the electrochemical detection of cancer, e.g., bladder cancer and prostate cancer, was focused largely on determining the concentration of one or more specific analyte(s). For example, Pursey et. al [Microfluidic electrochemical multiplex detection of bladder cancer DNA markers. Sensors Actuators, B Chem. 251, 34-39 (2017)] reported DNA-based probe with free-base porphyrin tag immobilized onto an array of electrodes which detects three BC DNA and changes the current signal after the hybridization with the probe onto the electrode surface. Other examples of electrochemical sensors for the identification of BC that lean on a “lock and key” mechanism include electrochemical immunoassays-based sensors [Wu, D. et al. Sensitive electrochemical immunosensor for detection of nuclear matrix protein-22 based on NH2-SAPO-34 supported Pd/Co nanoparticles. Scientific Reports vol. 6 (2016); Giannetto, M. et al., Competitive amperometric immunosensor for determination of p53 protein in urine with carbon nanotubes/gold nanoparticles screen-printed electrodes: A potential rapid and noninvasive screening tool for early diagnosis of urinary tract carcinoma. Anal. Chim. Acta (2017); Lee, M. H. et al. Electrochemical sensing of nuclear matrix protein 22 in urine with molecularly imprinted poly(ethylene-co-vinyl alcohol) coated zinc oxide nanorod arrays for clinical studies of bladder cancer diagnosis. Biosens. Bioelectron. 79, 789-795 (2016); Song, S. et al. Sensitivity Improvement in Electrochemical Immunoassays Using Antibody Immobilized Magnetic Nanoparticlesss with a Clean ITO Working Electrode. Biochip J. 14, 308-316 (2020); Song, S. et al. Electrochemical Immunoassay Based on Indium Tin Oxide Activity Toward a Alkaline Phosphatase. Biochip J. 13, 387-393 (2019)].

A similar approach of determining the level of a specific analyte in a biofluid sample, namely, a biomarker associated with the cancer under consideration, can also be found in the patent literature. For example, in WO 2014/032044, it was shown that alpha-methylacyl-CoA racemase, an enzyme that is overexpressed in prostate cancer epithelium and hence can serve as a potential biomarker for cancer cells within the prostate gland, can be quantified voltammterically using a biosensor consisting of a three-electrode configuration (counter electrode, reference electrode and one working electrode that was made of iridium oxide and active carbon powder).

In voltammetry, the current at the working electrode is measured as the potential applied across the working electrode and the counter electrode is varied with time. When electroactive species are present in the tested sample, they undergo oxidation (or reduction) when the potential on the working electrode is sufficiently positive (or negative). The oxidation/reduction electrochemical reactions are manifested by an increase in the current (anodic or cathodic) measured; that is, creation of an electrochemical signal with magnitude and position characteristic of a given analyte.

Instead of using a conventionally designed sensor for voltammetry, in which an analyte is measured over a single, highly selective working electrode, one can use a “voltammetric electronic tongue”—a device employing an array consisting of a few working electrodes that are different from another, i.e., the working electrodes are made of different metals or are surface-modified in a different manner (coated with distinct types of films), as described, for example, by del Valle, M. Electronic tongues employing electrochemical sensors. Electroanalysis vol. 22 1539-1555 (2010). Thus, the sensor array has semi-selective electrodes and cross-response features to “sense” more information. Then, chemometrics models are used, to deal with interference, allowing simultaneously classifying and quantifying multiple analytes. Voltammetric electronic tongues have also been suggested for use in connection with measurements in biofluids [Saidi et al. Voltammetric electronic combined tongue with chemometric techniques for direct identification of creatinine level in human urine. Meas. J. Int. Meas. Confed. 115, 178-184 (2018); Pascual, L. et al. Detection of prostate cancer using a voltammetric electronic tongue. Analyst 141, 4562-4567 (2016), and co-assigned WO 2018/225058, where a few working electrodes, surface-modified by different types of coatings, were assembled to create an electrochemical sensor that determined levels of the neurotransmitter dopamine in urine samples.

### THE INVENTION

We have now found that an electrochemical signal measured in a urine sample, such as a voltammogram measured by differential pulse voltammetry, can serve as a holistic electrochemical marker to detect types of cancer whose “fingerprint” can be found in the urine, e.g., urologic cancer, to distinguish BC or PC patients not only from other cancer patients but also from patients with inflammation. The electrochemical signal was generated by the total of redox-active species present in the urine sample, namely antioxidants and oxidants and was recorded with the aid of voltametric electronic tongue. The working electrodes incorporated into the device include a combination of bare electrode(s) and surface-modified electrode(s), which were selected based on their partial selectivity and cross-reactivity towards the antioxidant and oxidant systems. That is, the electrodes sense the cumulative effect of all antioxidants and oxidants present in the urine sample. In the experimental work reported below, ascorbic acid was used as a marker of Total Antioxidant Capacity (TAC) and hydrogen peroxide was used as a marker of Total Oxidant Status (TOS), and the electrodes were tested with respect to their ability to quantify these two markers. When the voltammograms are processed by suitable chemometric models, it is possible to classify the corresponding sample as belonging to BC or PC patients. That is, even if the specific electroactive molecules released to urine system by the patient are not identified, it is still possible for a suitable combination of electrodes that are sensitive to TAC marker (such as ascorbic acid) and TOS marker (such as hydrogen peroxide) to get an electrochemical fingerprint of BC/PC patients from their urine sample.

Accordingly, the invention is primarily directed to a method of screening and/or monitoring a patient for cancer that is detectable in urine, comprising:
obtaining a urine sample from the patient;
acquiring an electrochemical signal generated jointly by redox molecules in the urine sample, wherein the signal is acquired with the aid of an array of surface-modified electrodes that is sensitive for antioxidant and oxidant, wherein the array optionally further comprises one or more bare electrodes;
optionally preprocessing the electrochemical signal, to obtain processed data;
applying trained chemometric model(s) to the processed or raw data; and
determining the presence of cancer in the patient and/or monitorable cancer-related features (such as recurrence and/or progression of the disease, effectiveness of treatment, tumor classification), after the chemometric model has classified the processed data according to its training data set.

The preferred electrochemical technique to record the fingerprint of BC/PC in urine samples is voltammetry (application of varied voltage and measurement of current), such as differential pulse voltammetry (DPV). The description that follows focuses on in vitro detection of urologic cancer by the testing of urine sample, but other types of cancer, e.g., lung cancer and colorectal cancer, which have been shown to be detectable in urine, are included within the scope of the invention. See Mallafré-Muro, C. et al. [Comprehensive Volatilome and Metabolome Signatures of Colorectal Cancer in Urine: A Systematic Review and Meta-Analysis. Cancers 2021, 13, 2534. https://doi.org/10.3390/cancers13112534], and Yang et al. [Journal of Nanobiotechnology (2023) 21:153 https://doi.org/10.1186/s12951-023-01908-0].

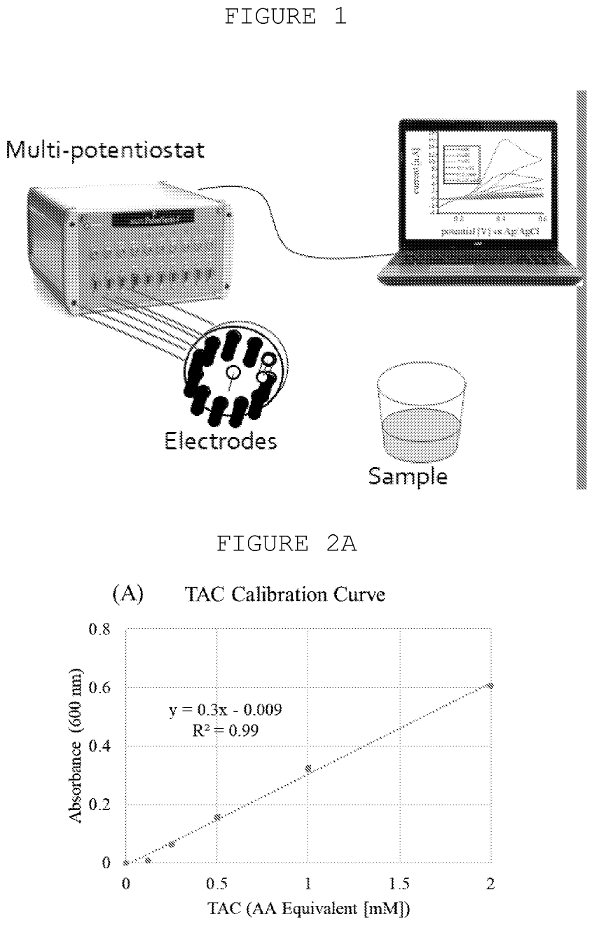

FIG. 1 shows the major components of the approach to urinary detection of BC or PC patients, namely, a urine sample that was taken from the patient and an array of electrodes to be inserted into the sample. The electrodes are connected to a potentiostat (which vary the potential across the electrodes to record a voltammogram). The voltammogram (or any other electrochemical signal) is resolved by the chemometric model to classify the urine sample as belonging to a specific type of urologic cancer.

In its most general form, the array of working electrodes is an assembly of a few sets of electrodes, each set consisting of electrode(s) of the same type. By electrodes of the same type we mean either bare electrode that are made of the same material (e.g., of the same noble metal), or electrodes coated with the same film material. The number of electrodes of type i is marked nᵢ. For example, five types (sets) of working electrodes are listed below (from which at least two sets, or at least three sets, can be chosen to create an array for voltammetry measurements in urine samples):
1) a set consisting of one or more bare electrodes. Bare electrodes are preferably made of noble metals, e.g., gold, platinum, rhodium, and iridium. Also, other electrodes, such as carbon electrodes, can be incorporated into the array of working electrodes. Gold is generally preferred, both for use as bare electrodes and surface-modified electrodes.
2) a set consisting of electrodes coated with polysaccharide (e.g., chitosan) film; typical film thickness is from 1 to 50 μm, e.g., 5 to 20 μm.
3) a set consisting of electrodes coated with polysaccharide (e.g., chitosan) film with conductive additives incorporated into the film, such as carbon nanotubes; typical film thickness is from 1 to 100 μm, e.g., 5 to 60 μm.
4) a set consisting of electrodes coated with reduced graphene oxide film; typical thickness is 200 to 1,000 nm, e.g., 350 to 550 nm.
5) a set consisting of electrodes coated with platinum black film; typical film thickness is from 1 to 50 μm, e.g., 6 to 10 μm.
   (coating thickness can be measured by atomic force microscopy or profilometry).

nᵢ—the number of electrodes in each set—is usually up to 3. In fact, because the volume of a urine sample may be limited, an efficient array design calls for a small number of working electrodes which could easily fit into a suitable electrochemical measurement cell, i.e., by selecting less than five types of electrodes and reducing the number of electrodes of each type (set). The experimental results shown below indicate that good results are obtained with a combination of three sets of electrodes consisting of bare (e.g., gold) electrodes (1≤n₁≤3); platinum black coated electrodes (1≤n₅≤3) and a third set which is either the set of chitosan-coated electrodes, the set of carbon nanotubes-added chitosan coated electrodes, or the set of reduced graphene oxide-coated electrodes {1≤nᵢ≤3; i=2, 3 or 4}.

But the electrochemical detection of BC/PC in urine samples is not limited to the use of electrodes coated with platinum black, reduced graphene oxide, chitosan, and CNT-added chitosan. The experimental work reported below shows how to apply suitable selection criteria so as to create an effective array with a fairly small number of bare and coated electrodes, based on the assumption that electrodes with good sensitivity towards ascorbic acid and hydrogen peroxide would accurately sense the total effect of all antioxidants and oxidants present in the urine sample, which in turn account for the existence of an electrochemical fingerprint of BC/PC cancer in urine samples.

Screening tests that can be applied to identify an effective combination I of bare and surface modified electrodes, from a group G of electrodes (I⊆G) are now described based, as mentioned above, on ascorbic acid (sometimes abbreviated herein AA) and H₂O₂; but it is possible to carry out similar screening tests to identify effective electrodes using other TAC and TOC candidate markers instead of ascorbic acid and H₂O₂, such as uric acid and thioredoxin.

First, the electrochemical performance of coated electrodes can be examined by cyclic voltammetry in ferrocyanide/ferricyanide redox couple [Fe(CN)₆³⁻↔Fe(CN)₆⁴⁻] solution—a benchmark frequently used to assess the acceptability of surface-modified electrodes.

Next, cyclic voltammetry can be used to assess the detectability of AA and H₂O₂ by each of the candidate working electrodes in G, by measuring the concentrations of AA and H₂O₂ (each separately) in PBS, across applicable concentration ranges, say, from 0.1 to 10 mM, to show linearity of current peak versus concentration curves. The slope of the linear equation fitted to the curve indicates the sensitivity of the tested electrode i towards the analyte (AA or H₂O₂), and is designated S_{i,AA} or S_{i,H2O2}. Working electrodes (bare or surface modified), with sensitivity above 3.0 mA/M, either towards AA (S_{i,AA}>3.0 mA/M) or H₂O₂ (S_{i,H2O2}>3.0 mA/M) are generally preferred for use in the sensor.

Partial selectivity is yet another useful selection criterion. Partial selectivity (PS) is calculated by dividing the sensitivity of electrode i towards ascorbic acid by the sensitivity of the same electrode towards H₂O₂. The ratio obtained is PS_{i,AA/H2O2} (or its inverse, PS_{i,H2O2/AA}):

$$P {S}_{i , AA / H 2 O 2} = \frac{{S}_{i , AA}}{{S}_{i , H 2 O 2}}$$

According to a preferred variant of the invention, the array used for the electrochemical detection of urologic cancer in a patient comprises at least one working electrode showing partial selectivity for ascorbic acid/hydrogen peroxide of above 5 (that is

$$\frac{{S}_{i , AA}}{{S}_{i , H 2 O 2}} > 5$$

and at least one working electrode showing partial selectivity for hydrogen peroxide/ascorbic acid of above 2 (that is

$$\frac{{S}_{i , H 2 O 2}}{{S}_{i , AA}} > 2.$$

For example, bare gold electrode and platinum black coated electrode show PS_{i,AA/H2O2} above 5 and PS_{i,H2O2/AA} above 2, respectively, and are therefore especially preferred for use in the invention. In fact, the platinum black-coated electrode seems to be quite unique in that it demonstrates increased partial selectivity towards hydrogen peroxide compared to the other electrodes.

Cross reactivity can then be calculated over a combination I consisting of, say, three different types of electrodes (I⊆G):

$$C {R}_{j} = \frac{1}{2} {\sum }_{i ∈ I} {\sum }_{k ∈ I} \vert P {S}_{i , j} - P {S}_{k , j} \vert$$

where i and k are two different types of electrodes in the combination I and j the target analytes. A combination I can then be rejected or accepted based on its CR. For example, for G={bare electrode, chitosan-coated electrode, chitosan+CNT coated electrode, Pt coated electrode and rGO coated electrode} useful three-electrode combinations I determined by the CR test were:
I1={bare electrode, Pt coated electrode, chitosan+CNT coated electrode};
I2={bare electrode, Pt coated electrode, chitosan coated electrode};
I3={bare electrode, Pt coated electrode, rGO coated electrode}.

A sensor based on the I1 combination (bare, Pt coated and chitosan+CNT coated electrodes) and a device comprising the sensor, a counter electrode and optionally reference electrode; a potentiostat/galvanostat, and a processor configured to analyze the electrochemical signal by one or more chemometric techniques, form additional separate aspect of the invention.

The coatings mentioned above (made of platinum black, reduced graphene oxide, chitosan, and chitosan to which conductive additive was added, such as carbon nanotubes) are applied onto surfaces of bare electrodes (e.g., on a 1 to 5 mm diameter disc shape commercial gold electrode) by a suitable electrodeposition technique:
(i) galvanostatic electrodeposition (chronopotentiometry), in which a constant current is passed through the electrode(s) to be coated;
(ii) potentiostatic electrodeposition (chronoamperometry), in which a constant potential is applied on the working electrode(s) to be coated; or
(iii) cyclic voltammetry electrodeposition.

Platinum black film can be generated via galvanostatic electrodeposition onto one or more electrodes, by passing a constant current (a cathodic current, with current density fixed in the range of 0.1 to 4 mA/cm², for 3 to 7 minutes; for example, a current density of 0.3 mA/cm² is supplied over five minutes), through a deposition solution in which a suitable platinum source is dissolved, e.g., by electrochemical reduction of chloroplatinic acid dissolved in DI water at a concentration in the range of 1% (w/w in water) to 3% (w/w in water), in the presence of about 0.05% (w/w in water) of lead acetate. Lead acetate enhances the electrode reaction (i.e., the reduction of Pt) in presence of platinum black solution and it also strengthens the adhesion of the coating to the electrode. The pH of the deposition solution is shifted to the strongly acidic by addition of hydrochloric acid. A two-electrode configuration can be used, which includes the electrodes to be coated as working electrode(s) and a ring or wire Pt counter electrode.

Another type of film-forming material that is applied to create film-coated microelectrode(s) is reduced graphene oxide. The deposition solution is prepared by known methods, e.g., the Hummers' method, where oxidation of graphite flakes or powder takes place upon adding the graphite to a cold solution of sulfuric acid (e.g., 0 °C) followed by gradual addition of sodium nitrate and potassium permanganate under continuous stirring. For example, on a laboratory scale, the addition time of each of the successively added NaNO₃ and KMNO₄ reagents is not less than ten to fifteen minutes. On completion of reagent's addition, the reaction mixture is heated to about 35-45 °C and kept under stirring for a couple of hours, e.g., not less than two hours. The reaction is terminated by addition of water and hydrogen peroxide which removes excess permanganate. The graphene oxide (GO) is recovered by centrifugation and freeze dried and used to prepare deposition solution with concentrations in the range from 0.1 to 0.9 mg/ml GO. A deposition solution can also be prepared by a modified Hammers procedure, which consists of adding the graphite powder (or flakes) to a mixed sulfuric acid/phosphoric acid solution (e.g., proportioned about 9:1 by volume), followed by the slow addition of KMnO₄. The mixture is kept under stirring for couple of hours at a slightly elevated temperature (at 30-35 °C) until the mixture acquires a dark green color. Termination of the reaction is achieved by slow addition of H₂O₂ aqueous solution (e.g., the commercial 30% w/w solution). Graphene oxide is recovered through acidification of the mixture by hydrochloric acid (e.g., addition of commercial 32% HCl solution and DI), centrifugation of the resulting solution, washing of the supernatant with HCl/water, drying of the washed solution (e.g., at 90 °C in an oven) and collecting the GO powder. The dried GO powder is dissolved in DI, usually up to concentration of 0.5 g/L GO concentration. Addition of an electrolyte to the GO solution affords the GO electrodeposition solution. Next, r-GO is obtained electrochemically from the GO solution onto the electrode (Au) surface, using cyclic voltammetry electrodeposition, in a three-electrode cell configuration consisting of the Au electrode(s) as working electrode(s); an externally applied Pt wire as counter electrode and Ag/AgCl as reference electrode. A potential window, for example from −1.4. to 1.4V (versus Ag/AgCl) is scanned at rate of in the range of to 50 to 500 mV/s, with number of cycles varying from 1 to 5.

Electrodeposited chitosan film-coated electrode can be prepared with the aid of a deposition solution with chitosan concentration in the range from 0.5 to 2.0 wt %, preferably from 0.8 to 1.2 wt %, prepared by dissolving chitosan in a strongly acidic environment, whereby the amino groups undergo protonation to reach a slightly acidic pH (5-6). As pointed out above, conductive additives can be included in the deposition solution; these additives will co-deposit and affect the film properties. The concentration of the additives in the deposition solution (e.g., carbon nanotubes (abbreviated herein CNT), gold nanoparticles and platinum nanoparticles) is in the range from 0.1 to 2%, preferably from 0.8 to 1.8 wt. %. For example, chitosan-CNT electrodeposition solution can be prepared by mixing a chitosan solution as previously described with CNTs, followed by ultra-sonication. The electrode is immersed in the chitosan deposition solution (or chitosan/CNT solution) and electrodeposition is achieved by the chronopotentiometry technique, i.e., selected electrodes to be coated are biased to the negative potential against a counter electrode with constant (cathodic) current being applied between the electrodes for a period of time of 0.5 to 5 min, supplied by a DC current source; typically the current is set in the range from 3 to 6 μA/cm². A two-electrode configuration can be used, i.e., the counter electrode is shorted to reference terminal. Weakly bound chitosan is removed from the microelectrode surface, by immersing the device in a buffer solution.

The electrochemical sensor could benefit from the incorporation of other types of surface-modified working electrodes, e.g., those described in WO 2022/137236, where coatings consisting of transition metal dichalcogenide (TMDC) of the formula MX₂, in which M is a transition metal (such as molybdenum and tungsten) and X is S or Se, are described. For example, MoS₂ and WS₂.

By TMDC^{A} and TMDC^{B}, for example, MOS₂^{A} and MOS₂^{B}, it is meant TMDC coatings electrodeposited on an electrode surface by the following methods:
TMDC^{A}: cycling a potential range A, which corresponds to the electrochemical double layer (EDL) potential region of the working electrode, at a scanning rate of at least 0.05 V·s⁻¹, e.g., from 1.0 to 10.0 V·s⁻¹;
TMDC^{B}: cycling a potential range B, which extends to more positive potentials than potential range A (extended EDL) at a scanning rate of at least 0.05 V·s⁻¹, e.g., from 0.05 to 2.0 V·s⁻¹.

For gold electrode, potential range A is above −0.4 V, e.g., from −0.3 to +0.7 V (vs Ag/AgCl) and potential range B is above −0.4 V and extends up to +1.3 V or up to +1.4 V (vs Ag/AgCl), e.g., from 0 to +1.4 V (vs Ag/AgCl). For carbon electrodes, the same potential ranges A and B can be used.

When the electrodes are made of gold, one way to differentiate between TMDC^{A}-coated electrodes and TMDC^{B}-coated electrodes is that the latter type shows the presence of hydroxide or oxide species associated with the metal electrode, e.g., for gold electrode, concomitant formation of a gold hydroxide adduct and the formation of a gold oxide layer occur (at +0.8 V vs Ag/AgCl and at +1.3 V vs Ag/AgCl in the extended EDL region, respectively), as determined, for example, by Raman spectroscopy or X-ray photon spectroscopy.

Another way to differentiate between TMDC^{A}-coated electrodes and TMDC^{B}-coated electrodes is that the former type shows lower capacitive currents (e.g., at least two-fold lower) and higher charge transfer resistance (e.g., at least ten fold-higher) than the latter type, as measured by electrochemical impedance spectroscopy (EIS) acquired by a solution of the ferrocyanide/ferricyanide redox couple [Fe(CN)₆³⁻↔Fe(CN)₆⁴⁻] by the technique described in detail in WO 2022/137236.

Thus, the sensor used in the present invention may include one or more MoS₂^{A}-coated electrodes, and/or one or more MOS₂^{B}-coated electrodes, and/or one or more WS₂^{A}-coated electrodes, and or one or more electrodes, WS₂^{B}-coated which were prepared by electrodeposition onto the surfaces of the working electrodes across potential ranges A and B, respectively, wherein potential range A corresponds to the double layer potential region of the working electrode and potential range B extends to more positive potentials than potential range A; such that, for example, for working electrodes made of noble metals, type B shows the presence of hydroxide form(s) and/or high oxidation state oxide forms(s) of the noble metal, whereas working electrodes of type A are free of such forms; and/or working electrodes of type B show higher capacitive currents and lower charge transfer resistance compared to working electrodes of subset A, as determined by electrochemical impedance spectroscopy. WO 2022/137236 describes the preparation of electrodeposition solutions and modification of the electrode surface to form:
MoS₂^{A}-coated electrode;
MoS₂^{B}-coated electrode;
WS₂^{A}-coated electrode; and
WS₂^{B}-coated electrode.

For example, the array of working electrodes used in the method of the invention comprises at least two sets of electrodes (e.g., 2-5 sets, or 6-8 sets, or 6-9 sets) selected from:
1) a set consisting of one or more bare electrodes;
2) a set of consisting of one or more electrodes coated with polysaccharide (e.g., chitosan) film;
3) a set consisting of one or more electrodes coated with polysaccharide (e.g., chitosan) film with conductive additives incorporated into the film;
4) a set consisting of one or more electrodes coated with reduced graphene oxide film;
5) a set consisting of one or more electrodes coated with platinum black film;
6) a set consisting of one or more electrodes coated with MoS₂^{A} film;
7) a set consisting of one or more electrodes coated with MoS₂^{B} film;
8) a set consisting of one or more electrodes coated with WS₂^{A} film; and
9) a set consisting of one or more electrodes coated with WS₂^{B} film;

The arrays of working electrodes, for example:
n_{bare}=n_{chitosan}=n_{chitosan+CNT}=n_{Pt}=n_{rGo}=2 (a total of ten electrodes);
n_{bare}=n_{chitosan+CNT}=n_{Pt}=2 (a total of six electrodes)
n_{bare}=n_{chitosan+CNT}=n_{Pt}=n_{rGo}=n_{MoS2A}=n_{MoS2B}=n_{WS2A}=n_{WS2B}=1 (a total of eight electrodes).
   may be arranged in different designs and geometries.

For example, one simple and straightforward configuration is based on the use of an electrochemical measurement cup to hold the urine sample (e.g., not less than 3 ml sample), fitted with a perforated cover; the holes in the cover correspond in number and size to the electrodes, such that individual working electrodes can be inserted into the measurement cup through the holes to be immersed in the sample. Commercial counter electrode (e.g., commercial Pt wire) and commercial reference electrode (Ag/AgCl) are also inserted into the cup. Such a design was used in the experimental work conducted in support of this invention and is described in more detail below.

A more sophisticated design is the one shown in WO 2018/225058. It was based on a cylindrical body made of silicon, polyvinyl alcohol or polydimethylsiloxane, which was 2 to 5 cm long and with diameter is in the range from 2 to 3 cm. The accessible surfaces of the electrodes were deployed on one base of the tubular body: a disc-shaped reference electrode positioned concentrically and coaxially in respect to the cylindrical body, a counter electrode at the vicinity of the reference electrode and multiple surface modified working electrodes (3.14 mm² each) positioned in radial direction from the reference and counter electrodes and evenly distributed along the perimeter of the base of the cylindrical body. The opposite base provides the electrical wiring to be connected to potentiostat/galvanostat (the electrodes extend along the cylindrical body and are connected to the wiring in the opposite base). When put to use, the electrochemical sensor is immersed in the urine sample to be analyzed such that the base of the cylinder, where the electrodes are disposed, is exposed to the sample allowing the electrodes that (optionally) protrude from the base to be dipped into the urine sample, creating the electrochemical cell for the measurements.

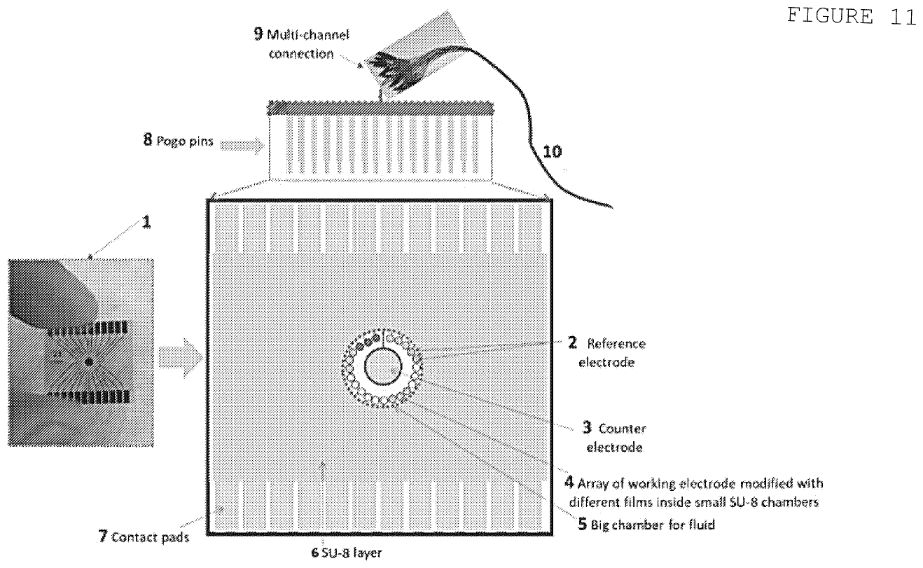

Alternative designs based on microfabricated configurations can also be considered for the electrochemical sensor. One example is shown in FIG. 11. An electrochemical sensor in the form of a microfabricated 1.5 cm×1.5 cm chip (1) on a glass substrate is shown. It can be a portable device or can be placed in the lab. The device dimensions are compatible with the conventional microfabrication techniques where the diameter of the working microelectrodes (4) is ~100 micrometer and the diameter of counter electrode (3) is ~500 micrometer. The chamber (5) is designed to hold small volume samples (10-30 microliter). Reference electrode (2) can be integrated into the array by electroplating one or two microelectrodes with Ag/AgCl as previously described (see Example 6 of WO 2022/137236). There are two kinds of chambers, a small chamber for each microelectrode opening (4 and 3) and a bigger chamber to carry the fluid sample (5). The chambers are made of insulating polymer, e.g., SU-8 polymer (6). The contacts pads (7) can be connected via pogo pins (8) and then to the multichannel connection (9) of the potentiostat or galvanostat unit (10 not shown). The device may be powered by a battery or alternatively, can be connected to a main power supply. A control unit (not shown) is designed to serve several purposes, chiefly controlling the potential of the working electrodes or the current flowing through the respectively, cell, according to the chosen electrochemical technique.

The microsensor described above (which consists of microelectrodes, microchambers encompassing the microelectrodes, all confined within a recessed zone that serves as a receptable for holding the liquid sample) can be created over a substrate by techniques such as etching and photolithography. Briefly, a substrate is cleaned, a first photoresist is applied (either negative, positive or image reversal resist), e.g., by spin coating, spray coating or dip coating, to produce a thin uniform layer on the substrate, followed by soft baking. A first mask is aligned, to transfer the pattern corresponding to electrodes' sites onto the surface of the substrate. The photoresist is exposed through the pattern on the mask with UV light, followed by a development step. Next, bare microelectrodes are deposited in the intended sites, e.g., first titanium which serves as an adhesion layer and then gold followed by lift off procedure that resulted in a gold microelectrode array on glass substrate. In order to define the electrode effective surface area, another lithography step was done using, e.g., SU-8 photoresist. To define the chamber for fluid, another photolithography step was followed with e.g., thick SU-8 resist.

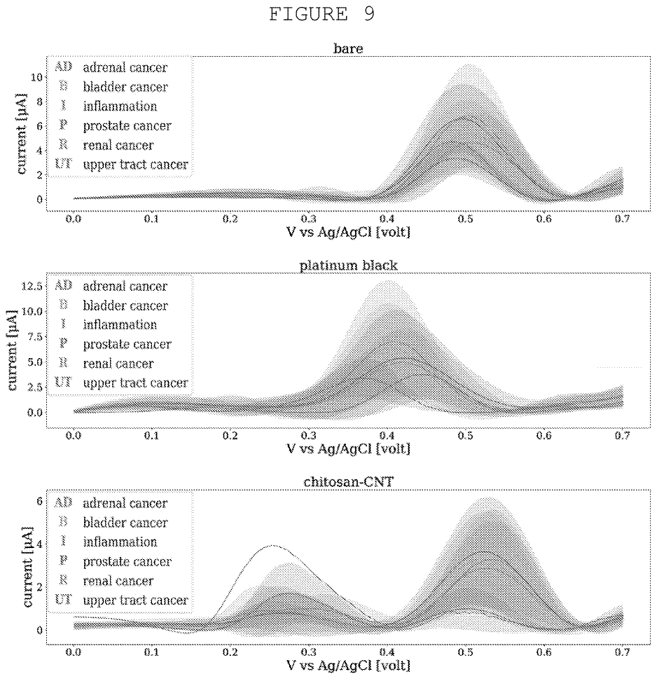

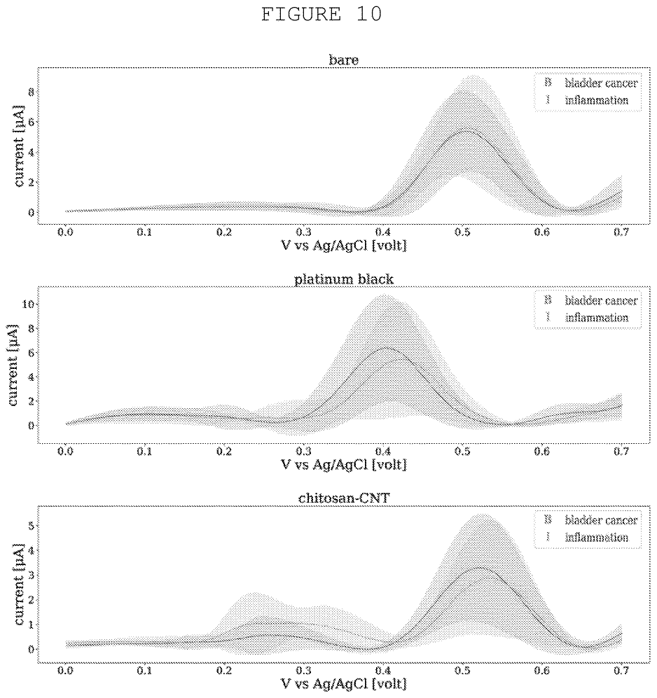

Having patterned the microstructures on the substrate, the desired coatings are applied on the gold microelectrodes, for example, by electrodeposition. The fabrication of such microsensors by photolithography and etching, followed by surface modification by electrodeposition, is described in detail in WO 2022/137236 (see FIGS. 9A, 9B, 9C and 10 and Example 6 of WO 2022/137236).

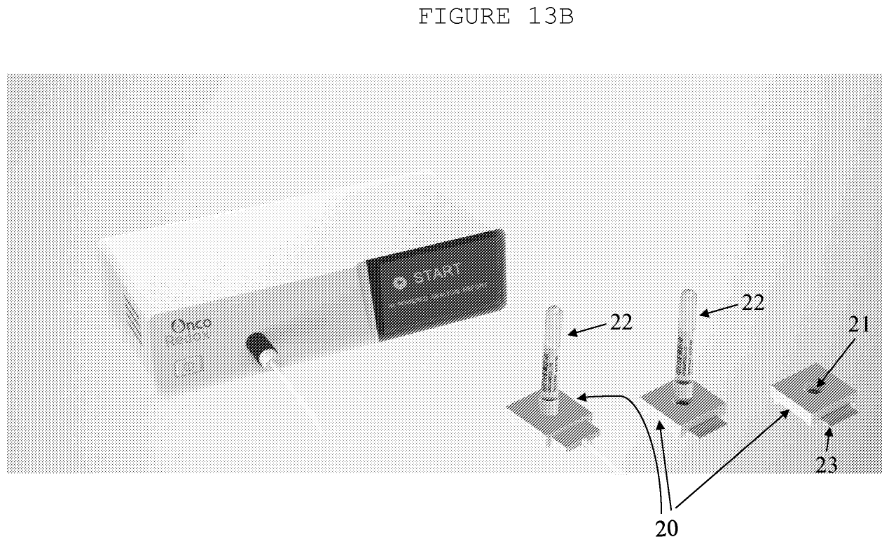

Another design with somewhat different geometry is shown in FIG. 13A. The counter electrode (labeled “AUX” for auxiliary) is ring-shaped, e.g., Pt ring with inner and outer diameters of 15 to 20 mm (e.g., 18 mm) and 21 to 25 (e.g., 22 mm), respectively. The reference electrode (labeled “REF”, e.g., Ag/AgCl coated electrode as previously described), is centrically positioned; it's surface area is about 30 to 40 mm² e.g., e.g., 36 mm². The working electrodes are placed in the annular space between the counter (outer) and reference (inner) electrodes, e.g., equally spaced apart from one another, and at distance of 4 to 8 mm, e.g., 6 mm from the center of the reference electrode. Each working electrode is disc-shaped with surface area of 25-30 mm², e.g., 27 mm². In the specific configuration of FIG. 13A, the following combination of working electrodes was assembled: a bare (gold or carbon) electrode, a chitosan/CNT-coated gold/carbon electrode, a platinum black-coated gold/carbon electrode, a reduced graphene oxide-coated gold/carbon electrode, a MoS₂^{A}-coated gold/carbon electrode, a MoS₂^{B}-coated gold/carbon electrode, WS₂^{A}-coated gold/carbon electrode and WS₂^{B}-coated gold/carbon electrode.

FIG. 13B is a photograph showing a small plastic housing (20) for the ring-shaped array of electrodes of FIG. 13A. A screen-printed-electrode (SPE) array with the geometry shown in FIG. 13A, consisting of non-coated carbon electrodes, is commercially available. The electrodes can be coated as previously explained and the surface-modified array is encased in the plastic housing (20), which is provided with a central circular opening (21) at its top. A urine sample container (22) can be fitted to the opening, to discharge the liquid sample onto the electrodes. The electrical connectors (23) extending from the bottom of the plastic housing are also shown.

In operation (for example, as shown in FIG. 13B) the electrodes are electrically connected to potentiostat or galvanostat which control the potential or current of the working electrodes, respectively, to create a data set of electrochemical signals when the electrodes are in contact with the test urine sample (the sample may be pretreated, i.e., steps such as freezing and thawing or centrifuging). The data set of electrochemical signals is analyzed by a processor applying one or more chemometric techniques.

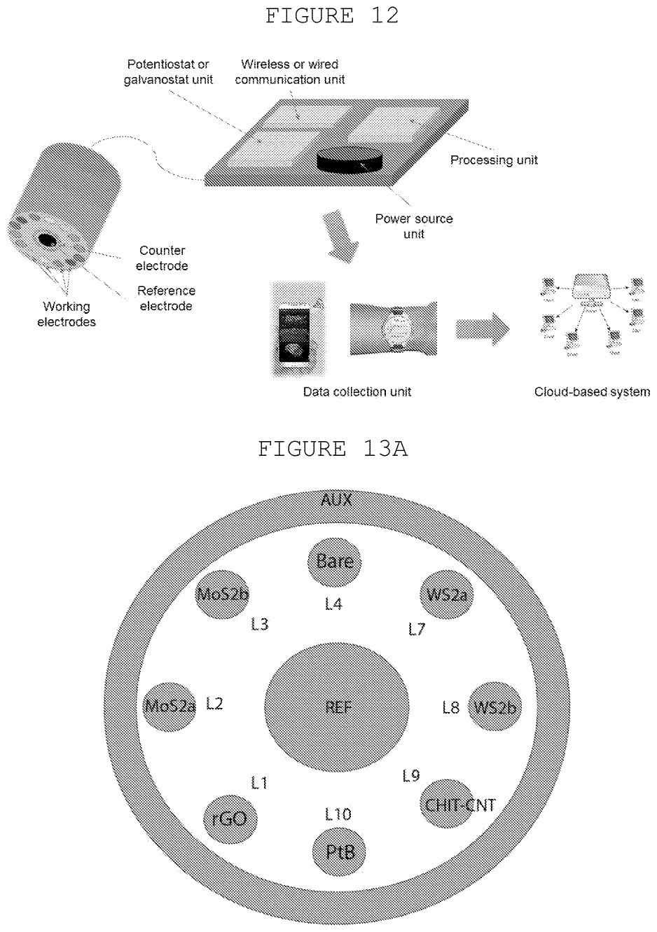

FIGS. 11-12 provide a schematic illustration of the electrochemical sensor according to the invention and a detection device into which the sensor is incorporated, i.e., either a portable device or a fixed device placed in a lab etc.

The device may further include a data storage unit or a data transmitting unit, i.e., wired transmitter or a wireless network transmitting unit with conventional communication ports to deliver the data to an externally located data storage unit.

A data storage unit may be the memory of the data processing unit or any computer readable media. In FIG. 12, personal instruments are shown and also a cloud-based data storage system.

The device further comprises a processor for analyzing a data set of electrochemical signals by one or more chemometric techniques, e.g., multivariate methods such as a supervised machine learning model (artificial neural network (ANN)), or a regression model, e.g. partial least square regression (PLSR).

Briefly, PLSR is a linear regression method and PLSR algorithms are available (e.g., MATLAB). As to ANN, a neural network model is generated with the aid of a training set. To this end, a matrix consisting of large number of samples with known concentrations of the analyte and with known outputs is collected. As explained in more detail below, the data set is split to create a training set, a cross-validation set and a test set. In the training process, the error between the outputs predicted by the neural network and the known outputs is calculated; this process continues, with the algorithm adjusting the parameters iteratively to minimize the error, i.e., to reduce the error below an acceptable level. Once created, the model is saved and can be used for future measurements of test samples.

It should be noted that raw test data collected by the electrochemical sensor undergoes pre-processing with the aid of known techniques before it is fed to the algorithm. Then methods such as principal component analysis (PCA), Fast Fourier Transform (FFT), and selection of important electrochemical signal features, can be used to reduce the dimensions of the data fed to the model. The latter method has been shown to be especially useful; the features selected (e.g., from the voltammograms) include peak current, peak potential, maximum slopes of the I vs. E function (for the increasing and decreasing parts of the function).

That is, to make a measurement of a test sample—using voltammetry for example—the sample is placed in the sample holder in contact with the electrochemical sensor in the device of the invention, as described above, varied voltage is applied by the potentiostat between the reference electrode and working electrodes, currents generated are measured and the measurements are stored, and the test data collected (readings from all working electrodes) is preprocessed, reduced and scaled, fed to the algorithm to obtain the model input.

The two approaches for model building-PLSR and ANN are now discussed in more detail; the major steps are outlined below. In both cases, data reduction is based on signal features.

### Model Building Process-Based Signal Samples (PLSR)

1. Organization of data in a cell structure—with the aid of MATLAB software reading csv files, all experimental data is arranged in one type of structure (e.g. cell type).

2. Signal smoothing—by using the signal processing toolbox, MATLAB software 2017a version, a built-in function (e.g. ‘filter’) can be used to filter the signals by employing a moving average window in order to reduce signal fluctuations and noisy behavior which is not originated by the electrochemical properties of the tested solution. A varied filter order in the range of 5<M<8, (M—filter order), depending on the noise level in the recorded data, can be used. In order to keep this parameter as unbiased for all the recorded signals in each experiment, it is kept fixed and equal to specific value for each experimental data.

3. Noise reduction due to size variation between different electrodes. In order to improve the signal to noise ratio (SNR), each electrode's signal is normalized by its electrode's effective surface area (A_{eff}). The electrode's effective surface area is obtained by experimental cyclic voltammetry with ferrocyanide and ferricyanide solution. The effective surface area of each electrode is calculated using the Randles-Sevcik equation

$${i}_{p} = 0.4 \; 46 {nFA}_{eff} C {( \frac{nFvD}{RT} )}^{\frac{1}{2}} ,$$

where iₚ is the current peak, n=1 is the total number of electrons transferred per molecule in the electrochemical process of ferrocyanide/ferricyanide, F is Faraday constant (96, 485.33 C mol⁻¹), A is the geometric area of the electrode/electrochemical area, C is the concentration of the electro-active species (10 μmol cm⁻³), D is diffusion coefficient of the analyte (6.95 cm² s⁻¹), v is scan rate (Vs⁻¹), R is universal gas constant (8.314 J K⁻¹ mol⁻¹) and T is temperature (°K). 4. Baseline subtraction—in an electrochemical analysis, the main interest is the faradaic current that is generated owing to the electron transfer from the redox molecule to the electrode surface in a specific electric potential (oxidation potential). In order to improve signal to noise ratio (SNR), the Asymmetric least squares spline regression (AsLSSR) was used. With the aid of MATLAB software 2017a version, a function is built to estimate the baseline signal by getting two constant values parameters, λ the smoothing parameter (10²<λ<10⁹) and p the asymmetry parameter (0.001<p<0.1). These two parameters take part in the numerical optimization of the cost function of the algorithm.

5. Organization of signals in a matrix structure—the signals are arranged in a matrix form, with each raw corresponding to specific array response. Signals were put in the matrix one after the other, to produce a super raw vector structure for each solution, while the target was defined as the concentration matrix, each column describing specific analyte concentration used through the experiments. This has been achieved by building MATLAB script (version 2017).

6. Dividing the data set into distinct subsets—the data is separated into two or three distinct sets. The first set is a training set, that is used for the training and the design of the model. All optimization procedures for finding the optimal solution are performed on the training set. It should be noted that the training set could be sub-divided to create a small cross-validation set. The other set is the test set. This set is used to check the model's generalization capabilities, by using the trained model in order to evaluate ability of the model to predict the concentrations in the “unseen” samples. The data is usually divided as follows: 70-85% of the samples are assigned to the training set (including ~10% that may be used for cross-validation) and 10-30% for testing. The samples are divided randomly, but the computer's random generation is fixed to assure that the same subdivision could be reproduced.

7. Signals centering—in order to focus on the variability of each specific potential, data is centered, checking the average features value for the all set, and subtract it from the all signal, resulted with features with mean value equal to 0. The average value of the training set is saved for future use for centering the test set.

8. Choosing a regression model for prediction analysis—the partial least square regression (PLSR) model, a linear technique, was used. It is especially suitable for cases where there is a high correlation between the different features and when there is a limited number of samples (e.g. solutions). The ‘plsregress’ MATLAB function toolbox was used for model building and testing.

9. Choose optimal model parameters (k-fold cross validation)—In order to choose wisely different digital (e.g. number of latent variable in a PLSR model) and physical parameters (e.g. electrode combination) the CV method (LOOCV and 10-fold CV) was used. With the aid of a code that is able to give all the possible configurations without repetition, the CV was implemented in the MATLAB software 2017a version, using the ‘cvpartition’ function from the statistical toolbox, for random divisions into k sets. By dividing the train set and using it also for validation we were able to take advantage of most of the information hidden in the data. Model parameters minimizing the cross-validation error were chosen.

10. Model training—the best number of latent variables and best electrode combinations were chosen for training the model on all the training set. A PLSR model using the ‘plsregeress’ function from MATLAB statistics tool box (2017 version) was built.

11. Test Data pre-processing—the test signals were centered according to the mean average value of the training set.

12. Model predictability—the trained model was used to test and evaluate the performance on unseen data set, i.e., the test set, which was preprocessed and was ready for use as the model input.

13. Evaluate model performance—the quality of the model is assessed with the root mean square error between the known concentrations and those that were estimated by the model.

$$R M S {E}_{test} = \sqrt{\frac{1}{{N}_{test}} {\sum }_{i = 1}^{{N}_{test}} {( {C}_{expected} - {C}_{calculated} )}^{2}}$$

(N is the number of samples; C_{expected} is the real actual value and C_{calculated} is the predicted value).

### Model Building Process-Based Direct Electrochemical Features (ANN)

1. organization of data in a cell structure—with the aid of MATLAB software, csv files are read, in order to arrange all the experimental data in one type of structure (e.g. cell type).

2. Signal smoothing—by using the signal processing toolbox, MATLAB software 2017a version, a built-in function (e.g. ‘filter’) is used to filter the signals by employing a moving average window in order to reduce signal fluctuations and noisy behavior which is not originated by the electrochemical properties of the tested solution. A varied filter order in the range of 5<M<8, (M—filter order), depending on the noise level in the recorded data, is used. In order to keep this parameter as unbiased for all the recorded signals in each experiment, it was kept fixed and equal to specific value for each experimental data.

3. Feature extraction—specific electrochemical signal features were extracted, i.e., features which are indicative of the identity of the redox-active molecule and its concentration in the solution. The extracted features include: peak potential, peak current, maximum slope of the signal, and current value at specific potentials (potentials which are known as the standard oxidation-reduction potential of specific analyte-good evaluation when the peak is not visible). All features extracted automatically using MATLAB software 2017a version built-in functions and by customary-built specific functions for each feature.

4. Organize features in a matrix structure—the extracted features were arranged in a matrix form, with each raw corresponding to specific array response, whereas each column describes specific analyte concentration through the experiment. This has been done by building MATLAB script (version 2017).

5. Dividing the data set into distinct subsets—The data is separated into two or three distinct sets. The first set is a training set, that is used for the training and the design of the model. All optimization procedures for finding the optimal solution are performed on the training set. It should be noted that the training set could be sub-divided to create a small cross-validation set, as explained below and further illustrated in the Examples below. The other set is the test set. This set is used to check the model's generalization capabilities, by using the trained model in order to evaluate ability of the model to predict the concentrations in the “unseen” samples. The data is usually divided as follows: 70-85% of the samples are assigned to the training set (including ~10% that may be used for cross-validation) and 10-30% for testing. The samples are divided randomly, but the computer's random generation is fixed to assure that the same subdivision could be reproduced.

6. Feature normalization—features were standardized using the z-score transformation (subtracting the mean value of each feature, and scaling it by dividing the value by the standard deviation). Scaling was preformed because the features were in different scales, such as peak currents [μA] and peak potentials [V]. The data transformation was achieved with the aid of MATLAB software 2017a version. The transformation was performed on the training set, when the moments value were saved for future scaling of the test data.

7. Feature selection—The strategy employed for data reduction to decrease computational complexity was ten-fold cross-validation forward selection based linear regression. The criterion for the selection was the root mean square error between the “real” concentration and those estimated for the validation set. This was achieved with the aid of the statistical toolbox of MATLAB software 2017a version. In each the experiments we used a different initial number of features depending on the technique that was chosen to extract data features.

8. Choosing regression model for prediction analysis—In order to perform multivariate analysis (not only one target value), artificial neural network (ANN) models were used—a nonlinear technique—to explore the relation between the extracted features to the analyte's concentration. The ANN MATLAB toolbox was used to explore different network architectures.

9. ANN model optimization (based k-fold cross-validation)—Simple ANN architectures, such as 1-hidden layer with limited number of neurons, was used in order to reduce the chance for overfitting—the lesser number of neurons in use the lower network complexity. The best architecture was chosen with the aid of a cross-validation test: the number of neurons in the hidden layer was varied to test the network performance on a validation set. The upper bound of the number of neuros was set such that it is smaller than the number of the model weights. Then the number of neurons with the best score (in terms of the root mean square error between the known concentration and those who were estimated on the validation set) was chosen. The test was repeated with different initial conditions (e.g., different weight initializations), because ANN models are significantly affected by their initial conditions; but in each individual test the parameters were fixed in order to make unbiased and robust decision.

10. Model training—having determined the best architecture, it was now used for training the model across the entire training set. The number of the training iterations was limited (early stopping) according to a specific error value that was set to stop the training procedure after reaching at least 99% of the target variance. Hence a trained network which minimizes the performance on the training data is created, ready for future testing.

11. Test data pre-processing—based on the selected features in the feature selection procedure, the test features were loaded and standardized according to the training moments. For each feature, the training mean value was subtracted and the result divided it by the training standard deviation (this procedure is based on the fact that the two sets sampled from the same data population), creating a scaled data set.

12. ANN predictability—The trained model was used to test and evaluate the performance on unseen data set, i.e., on the test set which was preprocessed and was ready for use as the model input. Calculations were performed in MATLAB software 2017a version, using the ANN toolbox function and aid function coded for specific tasks.

13. Evaluation of model performance—the quality of the model is assessed with the root mean square error (between the known concentrations and those that were estimated by the model:

$$R M S {E}_{test} = \sqrt{\frac{1}{{N}_{test}} {\sum }_{i = 1}^{{N}_{test}} {( {C}_{expected} - {C}_{calculated} )}^{2}}$$

(as previously defined) and the Pearson correlation coefficient (PCC):

$$P C C = \frac{E [ {C}_{expected} - {μ}_{expected} ] E [ {C}_{estimated} - {μ}_{estimated} ]}{{σ}_{expected}^{2} {σ}_{estimated}^{2}}$$

The metrics for classification models were:

Accuracy, which is defined by:

$$Accuracy = \frac{true positive + true negative}{total population}$$

Sensitivity, which is defined by:

$$Sensitivity = \frac{true positive}{p ositive}$$

False Positive Rate (FPR), which is as defined by:

$$FPR = \frac{fasle positive}{n egative}$$

Precision, which as defined as:

$$Precision = \frac{true positive}{true positive + false positive}$$

As shown below, seventy-two urine samples were collected from patients for creating the model (patients aged between 50 to 82; the group consisted of fifty-six men and fifteen women, with one patient without gender in the records):

TABLE 1

| disease | Number |
|---|---|
| adrenal cancer | 1 |
| Bladder cancer | 24 |
| Prostate cancer | 14 |
| Renal cancer | 21 |
| Upper tract cancer | 6 |
| Inflammation | 6 (five suspected as BC, |
| | one as an upper track cancer) |

For classification between BC and non-BC cancer patients and classification between PC and non-PC cancer patients, the data was split into train (90%) and test (10%) sets and validated with stratified 7-fold cross validation. ANN, with 1-8 extracted features selected for each electrode, emerged as the best model for differentiating BC patients or PC patients from non-BC or non-PC patients.

The invention relates to a method as described above for screening a patient for urologic cancer to determine, for a cancer patient or a subject suspected of having cancer, if the organ of cancer involvement is the urine bladder or prostate, wherein the chemometric model applied is an artificial neural network-based model that was trained with a training data set consisting of urine samples taken from bladder and prostate cancer patients, and optionally renal cancer, upper tract cancer patients and inflammation.

For example, 1-50 (e.g., 1-8) features are extracted for each electrode and from 1-50 (e.g., 1-20) nodes were applied in ANN.

For classification between BC patients and patients with inflammation, the data was split into train (80%) and test (20%) sets and validated with stratified 4-fold cross validation. PLS-DA model, with one extracted feature for each electrode, emerged as the best model for differentiating BC patients or PC patients from non-BC or non-PC patients.

Thus, the invention relates to a method as described above for screening a patient to determine if a patient has bladder cancer or inflammation, wherein the chemometric model applied is PLS-DA that was trained with a training data set consisting of urine samples taken from bladder cancer patients and patients having inflammation. At least one feature extracted for each electrode.

In addition to screening and diagnosing, the method of the invention can be used for cancer monitoring. By monitoring is meant, for example, tracking the recurrence and/or progression of cancer, classification of tumor according to WHO (World Health Organization) or EAU (European Association of Urology) guidelines, and effectiveness of treatment.

That is, the invention provides a method of monitoring a patient concerning a cancer detectable in urine, comprising:
obtaining a urine sample from the patient;
acquiring an electrochemical signal generated jointly by redox molecules in the urine sample, wherein the signal is acquired with the aid of an array of surface-modified electrodes that is sensitive for antioxidant and oxidant, and optionally a bare electrode;
optionally preprocessing the electrochemical signal, to obtain processed data;
applying trained chemometric model(s) to the processed or raw data; and
determining recurrence and disease progression, tumor classification as explained above, and/or treatment response, after the chemometric model has classified the processed data according to its training data set.

In the drawings:

FIG. 1 shows the major components of the approach to urinary detection of BC or PC patients, namely, a urine sample that was taken from a patient and an array of electrodes to be inserted into the sample.

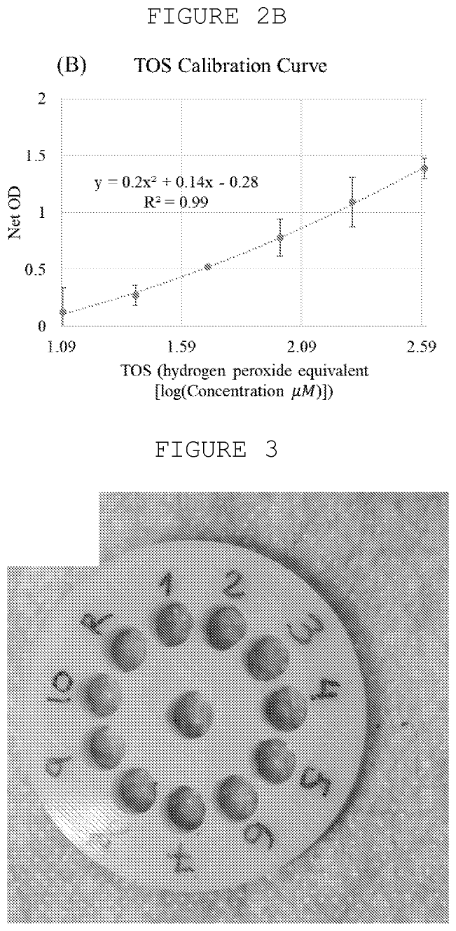

FIG. 2 shows a linear relationship between the AA concentration and the absorbance at 600 nm. FIG. 2B shows that polynomial relationship was observed between the log (concentration) to the net optical density (OD).

FIG. 3 shows the 3D printed cell cap, which was used for the measurements.

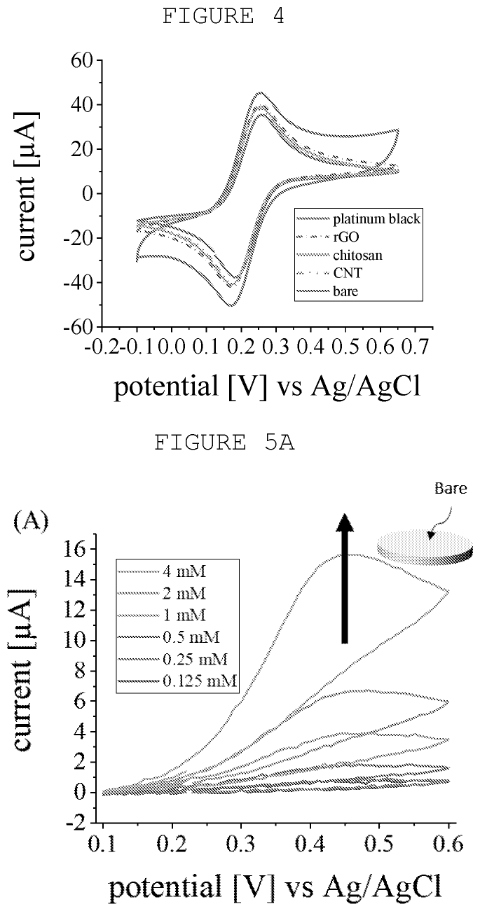

FIG. 4 is a voltammogram showing the detectability of the [Fe(CN)₆³⁻↔Fe(CN)₆⁴⁻] redox reaction by the bare and surface-modified electrodes.

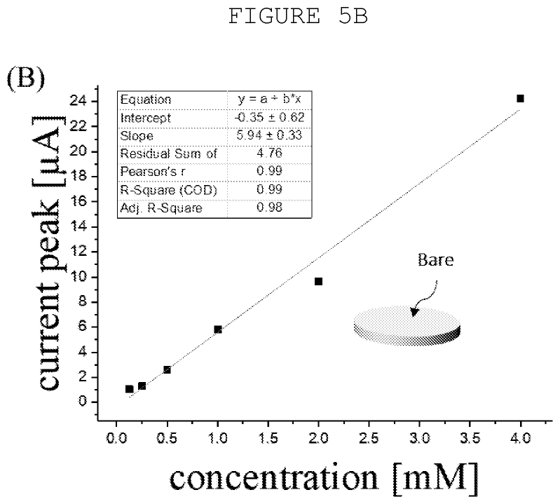

FIGS. 5A and 5B show current peak versus concentration plots based on the signals recorded by the bare electrode in ascorbic acid solutions with varying concentrations, and the calibration curve showing linear relationship.

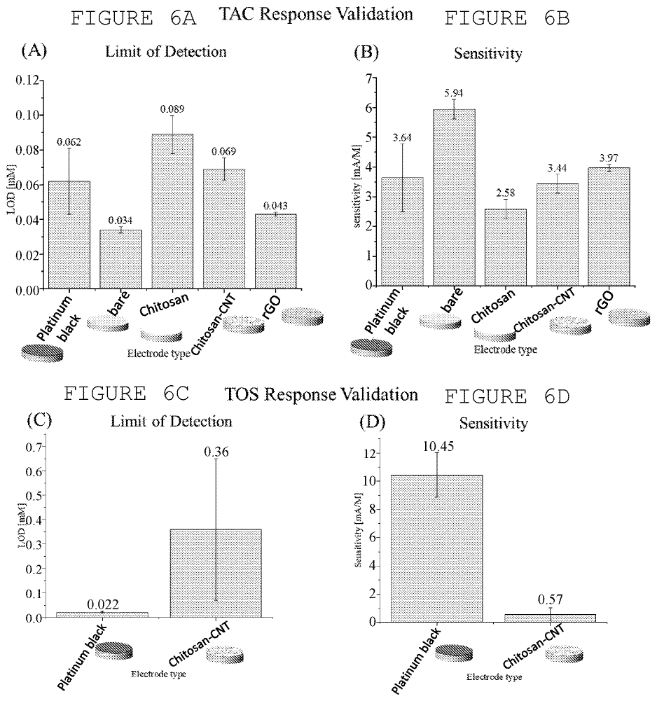

FIGS. 6A-6D show LOD and sensitivity in the form of bar diagrams.

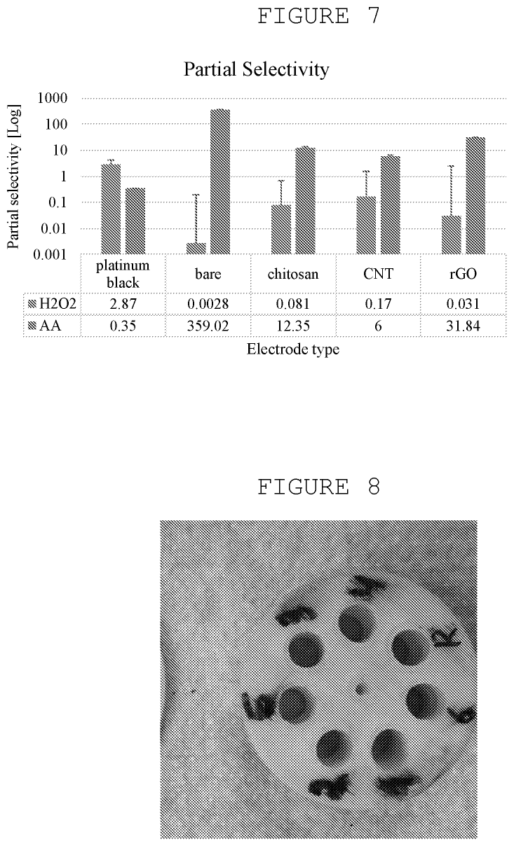

FIG. 7 is a bar diagram, showing partial selectivity for each one of the five types of electrodes used, in relation to ascorbic acid and hydrogen peroxide.

FIG. 8 shows a cell cap with the six-electrode array design (n_{bare}=n_{chitosan+CNT}=n_{rGo}=2)

FIGS. 9 and 10 show the voltammograms recorded by DPV of the urine samples.

FIG. 11 shows a design of a microsensor.

FIG. 12 shows detection device into which an electrochemical sensor is incorporated.

FIG. 13A shows a design of an array of electrodes and FIG. 13B is a photograph showing a plastic housing encasing the electrode array.

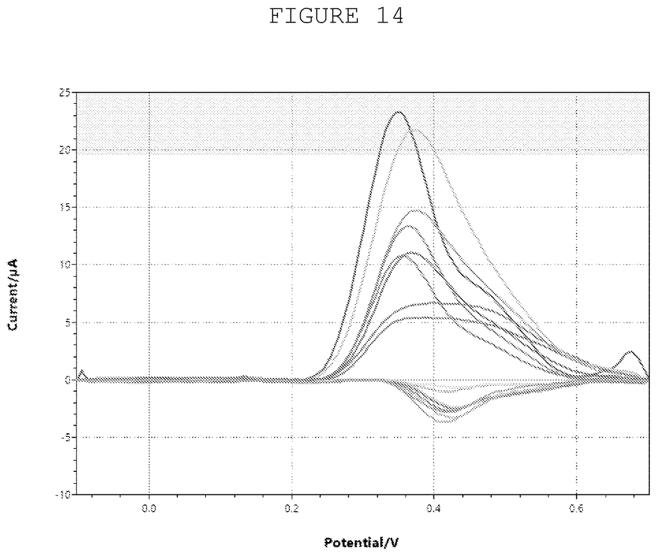

FIG. 14 shows the 2-ways voltammograms recorded by DPV of the urine samples using the modified commercial screen-printed-electrode (SPE) with 8-working carbon electrodes of FIG. 13A.

### EXAMPLES

### Preparations 1-6

### Electrodeposition Solutions

### 1) Chitosan Electrodeposition Solution (1 wt. %)

9 g chitosan powder was dissolved in 500 ml DDW and stirred for 20 min. 10 ml 2M HCl solution was added to the solution to reach pH of 5.5. The solution was sonicated for 45 minutes and then stirred again for 90 minutes at 700 rpm. The solution was filtrated with a metallic mesh 0.2 mm cylinder.

### 2) Chitosan-Carbon Nanotube Electrodeposition Solution (1 wt. %)

200 mg carbon nanotube, multi walled, was added to 20 ml of the 1 wt. % chitosan solution and stirred for 15 minutes in 250-500 rpm. Then the solution was sonicated for one hour. The solution was used during a storage period of one week.

### 3) Platinum-Black Electrodeposition Solution

Platinum black deposition solution was prepared by mixing 0.5 g of chloroplatinic acid and 25 mg of lead acetate in 50 ml of DI water. The mixture was then stirred and 3.9 μL of concentrated hydrochloric acid (32%; 10.2 Molar concentration) was added to the solution. The prepared solution was covered with aluminum foil and stored at room temperature.

### 4) Graphene Oxide Electrodeposition Solution

10 ml of 1 mg/mL rGO electrodeposition solution was prepared by diluting 2.5 ml of graphene oxide 4 mg/mL solution, with NaCl 100 mM in 5.5 ml DDW. The graphene oxide (GO) solution was prepared using a modified Hummers' method. A 9:1 ratio of sulfuric acid and phosphoric acid (100 mL) was prepared and stirred for several minutes. A graphite powder (7.5 g/L, 1 wt. eq.) was added to the mixture under stirring conditions. Potassium permanganate (45 g/L, 6 wt. eq.) was slowly added to the solution and the mixture was stirred for 6 h at 30-35 °C until the color turned to dark green. To eliminate the excess of potassium permanganate, hydrogen peroxide 30% w/w (2.5 mL) was added slowly and the mixture was stirred for 10 min, resulting in an exothermic reaction that was left to cool at room temperature. Concentrated 32% hydrochloric acid and DI were sequentially added at a 1:3 volume ratio and the resulting solution was centrifuged at 7000 RPM for 5 min. Residuals of the centrifuged solution were washed 3 times with hydrochloric acid and DI (1:3 v/v). The washed GO solution was dried at 90 °C in an oven (Binder-9010-0082) overnight, yielding the GO powder.

### 5) MoS₂ Electrodeposition Solution

200 ml of 0.1 mg/ml Molybdenum Disulfide 0.1M H₂SO₄ electrodeposition solution was prepared by diluting 20 ml 1.0 mg/ml Molybdenum Disulfide solution, with 0.98 ml H₂SO₄ in 179.02 ml DDW. The mixture was sonicated for 10 min.

### 6) WS₂ Electrodeposition Solution

200 ml of 0.1 mg/ml Tungsten Disulfide 0.1M H₂SO₄ electrodeposition solution was prepared by diluting 20 ml 1.0 mg/ml Tungsten Disulfide solution, with 0.98 ml H₂SO₄ in 179.02 ml DDW. The mixture was sonicated for 10 min.

### Preparation 7

### Fabrication of the Electrochemical Biosensor

### Surface Modification of Electrodes

2 mm diameter commercial gold electrodes were used (CH Instruments). Prior to coating, the surface of the electrode was polished by using a series of 1.0, 0.3, and 0.05 μm α-Al2O3 slurry on a micro-cloth pad until a mirror-shiny surface was obtained. The polished electrode was further rinsed with double-distilled water. The electrochemical activity of the polished electrode was validated after each polishing procedure by cyclic voltammetry (CV) measurement in 5 mM ferrocyanide/ferricyanide (ferro/ferri) solution to get signal as clean commercial electrode (0.037 mA). For validation by CV the next parameters were used: potential range −0.1V to 0.57V, scan rate 50 mV/sec. Then each electrode was rinsed with double distilled water (DDW) and coated with appropriate coating.

The following coating were electrodeposited onto the gold electrodes with VSP-300 Biologic potentiostat and EC-Lab software:

### Chitosan-Coated Electrodes

A chronopotentiometry technique was employed to electrodeposit chitosan from the solution of Preparation 1 onto gold electrodes over 300 s, at cathodic current density of 6 A/m², using a two-electrode configuration (a platinum wire as a counter electrode, and the gold as a working electrode). The modified electrodes were rinsed in double-distilled water (milli-Q, 18 MΩ) to remove chitosan that was not bound to the electrode.

### Chitosan-Carbon Nanotubes Coated Electrodes

A protocol akin to the one described for the chitosan electrodeposition was used to form chitosan-CNT coatings on gold electrodes (see Preparation 2 for the electrodeposition solution).

### Platinum-Black Coated Electrodes

A protocol akin to the one described for the chitosan electrodeposition was applied, but this time a cathodic current density of 0.3 mA/mm² was passed through the electrodeposition solution.

### Reduced Graphene Oxide Coated Electrodes

Cyclic voltammetry (CV) technique was employed for the electrodeposition, cycling 5 times across the potential range of −1.4 V to 1.4 V (vs. Ag/AgCl), at a scan rate=0.05 V/s. A three-electrode cell configuration consisting of the gold electrode (working electrode; ‘WE’), an externally applied commercial Pt wire (CHI115, CH Instruments; counter electrode; ‘CE’), and a Tungsten needle (P/N H-20242, Quarter) coated with Ag/AgCl ink (011464, BAS Inc.; pseudo reference electrode; ‘RE’).

### Assembling Electrode Array

Two different array designs were prepared and tested:

The first design consisted of ten working electrodes divided equally into five sets as follows:
n_{bare}=n_{chitosan}=n_{chitosan+CNT}=n_{Pt}=n_{rGo}=2

The second design consisted of six working electrodes divided equally into three sets as follows:
n_{bare}=n_{chitosan+CNT}=n_{Pt}=2

The geometrical arrangement of the electrodes is illustrated in the experimental setups that are shown in the next set of Examples. The electrodes were inserted into the solution/sample in an electrochemical cell through the holes of a suitably perforated cell cap; the set of working electrodes and one reference electrode were arranged along the circumference of a circle encircling a centrically positioned counter electrode.

### Preparation 8

### Fabrication of the Electrochemical Biosensor

### Surface Modification of Electrodes

A commercial screen-printed-electrode (SPE) with 8 working carbon electrodes of 2.95 mm diameter was used (Metrohm DropSens). The working electrodes were sharing a silver reference electrode, and a carbon counter electrode. The SPE substrate material was ceramic. The geometrical arrangement of the electrodes is illustrated in FIG. 13A.

Prior to coating, the surface of the electrode was cleaned by cyclic voltammetry using 0.5M H₂SO₄ sulfuric acid. The electrodes were further with rinsed double-distilled water. The electrochemical activity of the electrodes was validated by cyclic voltammetry (CV) measurement in 5 mM ferrocyanide/ferricyanide solution. Then each electrode was rinsed with double distilled water (DDW) and coated with appropriate coating.

The following coating were electrodeposited onto the carbon electrodes with Palmsens4 potentiostat:

### Platinum-Black Coated Electrodes (FIG. 13A, Working Electrode 1, Pin 10):

A chronopotentiometry technique was employed to electrodeposit platinum black from the solution of Preparation 3 onto carbon electrode over 5 mins, at cathodic current density of 4.8 mA/cm², using commercial platinum wire and Ag/AgCl reference electrodes (ASL, Japan). The modified electrodes were rinsed in double-distilled water (milli-Q, 18 MΩ) to remove residuals.

### Reduced Graphene Oxide Coated Electrodes (FIG. 13A, Working Electrode 2, Pin 1)

Cyclic voltammetry (CV) technique was employed for the electrodeposition, cycling 3 times across the potential range of −1.4 V to 1.4 V (vs. Ag/AgCl), at a scan rate=0.1 V/s. A commercial Ag/AgCl reference electrode (ALS, Japan) was used.

### Molybdenum Disulfide Type A-Coated Electrodes (FIG. 13A, Working Electrode 3, Pin 2):

Cyclic voltammetry (CV) technique was employed for the electrodeposition from the solution of Preparation 5, cycling 400 times across the potential range of −0.3 V to 0.7 V (vs. Ag/AgCl), at a scan rate=1 V/s. A commercial Ag/AgCl reference electrode (ALS, Japan) was used.

### Molybdenum Disulfide Type B-Coated Electrodes (FIG. 13A, Working Electrode 4, Pin 3):

Cyclic voltammetry (CV) technique was employed for the electrodeposition from the solution of Preparation 5, cycling 400 times across the potential range of 0 V to 1.4 V (vs. Ag/AgCl), at a scan rate=1 V/s. A commercial Ag/AgCl reference electrode (ALS, Japan) was used.

### Carbon Electrode (FIG. 13A, Working Electrode 5, Pin 4)

Carbon electrode was left untreated. The carbon electrodes were rinsed in double-distilled water (milli-Q, 18 MΩ) to remove residuals.

### Tungsten Disulfide Type A-Coated Electrodes (FIG. 13A, Working Electrode 6, Pin 7):

Cyclic voltammetry (CV) technique was employed for the electrodeposition from the solution of Preparation 6, cycling 400 times across the potential range of −0.3 V to 0.7 V (vs. Ag/AgCl), at a scan rate=1 V/s. A commercial Ag/AgCl reference electrode (ALS, Japan) was used.

### Tungsten Disulfide Type B-Coated Electrodes (FIG. 13A, Working Electrode 7, Pin 8):

Cyclic voltammetry (CV) technique was employed for the electrodeposition from the solution of Preparation 6, cycling 400 times across the potential range of 0 V to 1.4 V (vs. Ag/AgCl), at a scan rate=1 V/s. A commercial Ag/AgCl reference electrode (ALS, Japan) was used.

### Chitosan-Carbon Nanotubes Coated Electrodes (FIG. 13A, Working Electrode 8, Pin 9)

A chronopotentiometry technique was employed to electrodeposit chitosan+CNT from the solution of Preparation 2 onto carbon electrode over 5 mins, at cathodic current density of 0.595 mA/cm², using commercial platinum wire and Ag/AgCl reference electrodes (ASL, Japan). The modified electrodes were rinsed in double-distilled water (milli-Q, 18 MΩ) to remove residuals.

### Example 1

### Response Validation

### Part A: Spectrophotometric Response Validation

### Reagents and Solutions

TAC assay based on the ABTS (2,2′-Azino-bis(3-ethylbenzothiazoline-6-sulfuric acid) test: the first reagent is 0.4M acetate buffer solution (pH 5.8) that contains sodium acetate solution (470 ml) and acetic acid solution (30 ml). The acetate solution contains 16.4 g sodium acetate dissolved in 500 mL of ultrapure water. The acetic acid solution contains 1.1484 ml of reagent-grade glacial acetic acid that was added to 48.8516 ml ultrapure water. The second regent is ABTS radical in acetate buffer (30 mM, pH 3.6). The acetate buffer solution contains 37.5 mL sodium acetate solution that was mixed with 462 mL of the acetic acid solution. The sodium acetate solution contains 1.23 g sodium acetate dissolved in 500 mL of ultrapure water. The acetic acid solution contains 861.3 μL of reagent-grade glacial acetic acid was added to 499.1387 mL of ultrapure water. Then, 8.0833 μL of H₂O₂ was diluted in 25 ml acetate buffer (described above). Then 0.1372 g of ABTS was dissolved in the prepared solution.

TOS assay based on the Fe³⁺ ion and xylenol orange: the first reagent contains 150 μM xylenol orange, 140 mM NaCl, and 1.35 M glycerol. A 250 ml solution of the reagent was prepared from 0.0285 g of xylenol orange, 2.045 g of NaCl, 225 mL of 25 mM H₂SO₄ and 25 mL of glycerol. The second reagent contains 5 mM Ferrous ammonium sulfate, and 10 mM o-dianisidine dihydrochloride. For preparation of 250 mL solution of the second reagent, 0.49 g of (NH₄)₂Fe(SO₄)₂·6H₂O and 0.6107 g of o-Dianisidine dihydrochloride were dissolved in 250 mL of 25 mM H₂SO₄. 25 mM H₂SO₄ was obtained by diluting 25 mL of 0.5 M H₂SO₄ in 475 mL of ultrapure water. The concentrated 0.5 M H₂SO₄ solution was obtained by adding 679.3 UL of H₂SO₄ 18.2M to 24.321 mL DI Water. The solutions were stored for maximum 6 months at 4 °C.

Ascorbic acid solutions: Ascorbic acid solution was prepared at concentration of 4 mM. Then, it was serially diluted in ultrapure water and phosphate buffer saline (PBS) across the range of 0.125 mM up to 4 mM for the calibration curve performance of the spectrophotometric TAC assay and the electrochemical response validation.

H₂O₂ solutions: Two hydrogen peroxide solutions were prepared at concentrations of 4 mM and 400 μM. Then, each of these solutions was serially diluted in ultrapure water and PBS over the range of 0.125 mM up to 4 mM and 12.5 μM up to 400 μM for the calibration curve performance of the spectrophotometric TOS assay and the electrochemical response validation.

The PBS (phosphate buffer saline) was prepared by dissolving 1779.9 mg sodium phosphate dibasic, 244.96 mg sodium phosphate monobasic, 8.006 g sodium chloride and 201.28 mg potassium chloride in 1 L of deuterium-depleted water (DDW), to obtain for 10 mmol/L solution at pH 7.4.

### Spectrophotometric Measurements

Determination of TAC: a 12 μL of a sample and 250 μL of reagent 1 were added to a 96-well plate and incubated for 2 minutes. Then, the absorbance was measured at 600 nm. After that, 25 μL of reagent 2 was added to the reaction. After 5 minutes incubation, the second measurement was taken. The difference between the first to second measurements was used to calculate TAC in all assays. The results were expressed in millimoles of ascorbic acid equivalents per liter.

The TOS method is based on a colored complex formed between the ferric ion and xylenol orange in an acidic medium. For TOS assay, briefly, first, 35 μL of sample and 225 μL of reagent 1 were added to a 96-well plate and incubated for 2 minutes. Then, the absorbance was measured at 560 nm followed by secondary measurement at 800 nm. After that, 11 μL of reagent 2 was added to the reaction. After 5 minutes incubation the third and fourth measurements were taken at the same wavelengths. The difference between the first to the second measurements and between the third to the fourth measurements were used to calculate TOS in all assays. The assays results were expressed in micromoles of hydrogen peroxide equivalents per liter.

### Results

FIG. 2A shows that linear relationship was observed between the AA concentration and the absorbance at 600 nm, as expected (the higher the AA concentration, the higher the absorbance at 600 nm). FIG. 2B shows that polynomial relationship was observed between the log (concentration) to the net optical density (OD). In the literature, exponential relationship was observed, nevertheless, polynomial curve led to a better score in terms of R-squared values. After that, the measurements of the urine samples were fitted according to the calibration curves as a gold standard for the electrochemical sensor for OS measurements development.

Part B: Electrochemical Response Validation of the Ten-Electrode Array Design (n_{bare}=n_{chitosan}=n_{chitosan+CNT}=n_{Pt}=n_{rGo}=2)

An experimental setup, that consisted of a 20 ml electrochemical cell fitted with a 3D printed cell cap, was used for the measurements. The cap is shown in FIG. 3. The cap is perforated with twelve holes, which include a centrically located hole for positioning Pt counter electrode and eleven holes arranged in a ring fashion encircling the Pt counter electrode, to position the ten working electrodes (their holes are labeled by numbers 1 to 10) and Ag/AgCl reference electrode (its hole is labeled by the letter R). The electrodes inserted into the solution through the holes (1 mm in diameter) were connected to a potentiostat and a computer (Ivium potentiostat and IviumSoft software).

The experimental setup was used to characterize the electrochemical performance of the coated electrodes towards the [Fe(CN)₆³⁻↔Fe(CN)₆⁴⁻] benchmark (part B1) and markers associated with TAC (ascorbic acid) and TOS (hydrogen peroxide) (part B2).

B1: Cyclovoltammetry 5 mM solution of the in a ferrocyanide/ferricyanide redox couple [Fe(CN)₆³⁻↔Fe(CN)₆⁴⁻] was performed across the potential range of −0.1V to +0.65V, for a total of three cycles. The voltammogram is shown in FIG. 4, indicating the detectability of the [Fe(CN)₆³⁻↔Fe(CN)₆⁴⁻] redox reaction by the bare and surface-modified electrodes. Next, diluted solutions with concentrations spanning the range from 4 mM to 0.125 mM (dilution by factor ½) were prepared to create calibration curves for each type of electrode towards the [Fe(CN)₆³⁻↔Fe(CN)₆⁴⁻] analyte, to determine LOD of sensitivity of each electrode. LOD of electrode i {i=bare, chitosan, chitosan+CNT, Pt and rGO} towards analyte j {j=[Fe(CN)₆³⁻↔Fe(CN)₆⁴⁻]} was calculated by the formula:

$${LOD}_{i , j} = 3 \times \frac{PBS std}{{s}_{i , j}} \qquad ( 1 )$$

where PBS std is the standard deviation of the current peak of the analyte taken from 50 PBS samples and S_{i,j} denotes the sensitivity, which is the slope of the current versus concentration calibration curve, of electrode i toward analyte j. The results are tabulated in Table 2.

TABLE 2

| Electrode | Sensitivity [mA/M] | LOD [mM] |
|---|---|---|
| bare | 6.96 ± 0.15 | 0.021 ± 4.4 · 10⁻⁴ |
| chitosan | 5.84 ± 0.8 | 0.026 ± 3.56 · 10⁻³ |
| Chitosan + CNT | 9.71 ± 0.9 | 0.017 ± 1.63 · 10⁻³ |
| platinum black | 8.14 ± 0.9 | 0.022 ± 2.47 · 10⁻³ |
| rGO | 11.1 ± 0.38 | 0.015 ± 5.07 · 10⁻⁴ |

It is seen that the surface-modified electrodes, with the exception of the chitosan-coated electrode, show enhanced selectivity towards the [Fe(CN)₆³⁻↔Fe(CN)₆⁴⁻] analyte, compared to the bare electrode, and improved LOD can be achieved by the rGO and CNT-added chitosan modified electrodes, due to the conductive nature of the CNT, platinum black and rGO which account for amplification of the electrochemical signal. Surface roughness of the platinum black and rGO electrodes also contributes by increasing the capacity of the double layer, leading to additive current.

B2: Cyclovoltammetry in solutions of TAC marker (ascorbic acid) and separately in solutions of TOS marker (hydrogen peroxide) was performed across the potential range of −0.1V to +0.65V, for a total of three cycles. Diluted PBS solutions of ascorbic acid or hydrogen peroxide with concentrations spanning the range from 4 mM to 0.125 mM (dilution by factor ½) were prepared. Calibration curves that were generated based on the 0.125, 0.25, 0.5, 1.0, 2.0 and 4.0 mM solutions (current peak versus concentration plots) showed linear relationship, as indicated for the purpose of illustration in FIGS. 5A and 5B, for the signals recorded by the bare electrode in ascorbic acid solutions. The same procedure was applied for all the coated electrodes in respect of both ascorbic acid and hydrogen peroxide, to calculate the sensitivity S_{i,j} and the LOD_{i,j} for electrode i {i=bare, chitosan, chitosan+CNT, Pt and rGO} towards analyte j {ascorbic acid and H₂O₂}.

The LOD and sensitivity results are shown in the form of bar diagrams in FIGS. 6A (LOD) and 6B (sensitivity) for i=bare, chitosan, chitosan+CNT, Pt and rGO; j=ascorbic acid} and in FIGS. 6C (LOD) and 6D (sensitivity) for i=chitosan+CNT and Pt; j=hydrogen peroxide). It is seen that all surface-modified electrodes were sensitive towards ascorbic acid with platinum black-coated electrode emerging especially useful in detecting hydrogen peroxide.

Next, three parameters of the entire experimental set-up consisting of n_{bare}=n_{chitosan}=n_{chitosan+CNT}=n_{Pt}=n_{rGo}=2 were calculated. These parameters are:
Sⱼ, which is the average response slope towards analyte j, and is given by:

$${S}_{j} = \frac{1}{n} {\sum }_{i = 1}^{n} {S}_{i , j} \qquad ( 2 )$$

j is ascorbic acid or H₂O₂, S_{i,j} is the corresponding sensitivity as previously defined, and the summation is over the number of electrodes (n=10);
Kⱼ, which is the average signal-to-noise ratio (SNR) in relation to analyte j, and is calculated as follows:

$${K}_{j} = \frac{1}{n} {\sum }_{i = 1}^{n} {K}_{i , j} = \frac{1}{n} {\sum }_{i = 1}^{n} \frac{{S}_{i , j}}{{s}_{i , j}^{2}} \qquad ( 3 )$$

where

$${s}_{i , j}^{2}$$

is the standard deviation of the response slope, and the summation is over the number of electrodes (n=10);
Fⱼ, which is the non-selectivity factor in relation to analyte j, and is defined as:

$${F}_{j} = \frac{{S}_{j}}{{s}_{j}^{2}} \qquad ( 4 )$$

where Sⱼ is the average response slope, as defined in equation (2), and

$${s}_{j}^{2}$$

is its standard deviation.

The results for the two analytes are tabulated in Table 3, indicating the high sensitivity and stability of the electrode array with respect to the tested markers.

TABLE 3

| Analyte | ${S}_{j} = \frac{1}{n} {\sum }_{i = 1}^{n} {S}_{i , j}$ | ${K}_{j} = \frac{1}{n} {\sum }_{i = 1}^{n} \frac{{S}_{i , j}}{{s}_{i , j}^{2}}$ | ${F}_{j} = \frac{{S}_{j}}{{s}_{j}^{2}}$ |
|---|---|---|---|
| Ascorbic acid | 3.9 ± 1.1 mA/M | 14.8 ± 11.0 | 3.52 |
| H₂O₂ | 3.4 ± 3 mA/M | 17.0 ± 14.6 | 1.1 |

Next, partial selectivity (PS) was calculated:

$$P {S}_{i , j} = \frac{{s}_{i , j}}{{s}_{i , k}} \qquad ( 5 )$$

Partial selectivity is calculated by dividing the sensitivity of electrode i towards analyte j (ascorbic acid) by the sensitivity of the same electrode towards the other analyte k (H₂O₂), namely, by dividing the corresponding slopes of the calibration curves. The results are shown graphically in FIG. 7, in the form of bar diagrams, and in tabular form, for each one of the five types of electrodes used, in relation to ascorbic acid and hydrogen peroxide (namely, a pair of bars is assigned to each electrode type, though in fact PS_{i,j} is the inverse of PS_{j,i}). It is seen that the platinum black-coated electrode is unique in that it demonstrates increased partial selectivity towards hydrogen peroxide compared to the other electrodes.

The partial selectivity values were then used to calculate the cross reactivity (CR) over various electrode combinations I, each combination consisting of three electrode types from the group G of five electrode types:

$$C {R}_{I , j} = \frac{1}{2} {\sum }_{i ∈ I} {\sum }_{k ∈ I} \vert {PS}_{i , j} - P {S}_{k , j} \vert \qquad ( 6 )$$

The results are tabulated in Table 4:

TABLE 4

| | TAC marker | TOS marker |
|---|---|---|
| electrodes | Ascorbic acid | Hydrogen peroxide |
| chitosan, bare, CNT | 706.05 ± 10.9 | 0.33 ± 2.5 |
| bare, chitosan, rGO | 693.35 ± 11.5 | 0.16 ± 4.7 |
| bare, rGO, CNT | 706.05 ± 11.5 | 0.33 ± 4.7 |
| chitosan, CNT, rGO | 51.7 ± 2.4 | 0.27 ± 3.7 |
| platinum black, chitosan, CNT | 24 ± 1.79 | 5.58 ± 1.57 |
| platinum black, chitosan, rGO | 62.99 ± 2.4 | 5.67 ± 3.7 |
| platinum black, CNT, rGO | 62.99 ± 0.7 | 5.67 ± 2.2 |
| platinum black, bare, CNT | 717.35 ± 11 | 5.73 ± 2.5 |
| platinum black, bare, rGO | 717.35 ± 11.5 | 5.73 ± 4.7 |
| platinum black, bare, chitosan | 717.35 ± 10.9 | 5.73 ± 2.4 |

It may be desired to minimize the size of the electrodes array by reducing the number of electrodes employed, because sometimes only a limited volume of sample is available from patients for the tests. The set of operative electrodes may be reduced in number, e.g., three types instead of five, using the cross-reactivity data as a selection criterion. It is seen that useful ternary combinations of electrodes for detecting TAC and TOS markers comprise 1) platinum black-coated electrode and 2) bare electrode, whereas for the third electrode, one may choose from chitosan coated electrode, CNT-added chitosan coated electrode, and reduced graphene oxide-coated electrode, as all three types show roughly comparable contribution to the CR.

### Example 2

Voltammetry Measurements in Urine Samples of Patients Diagnosed with Cancer/Inflammation Using an Array of Working Electrodes and Signal Analysis with Chemometrics

The goals of the study were to differentiate between:
A) the redox fingerprints of BC or PC patients and patients diagnosed with other kinds of cancer; and
B) the redox fingerprints of BC patients and non-cancer patients with inflammation only.

### Urine Sample Preparation

5 to 10 ml urine samples were collected from seventy-two cancer patients. The group consisted of fifty-six men and fifteen women, with one patient without gender in the records. The patients were aged over fifty, and were diagnosed with adrenal cancer (n=1), BC (n=24), prostate cancer (n=14), renal cancer (n=21), upper tract cancer (n=6), and six patients with inflammation (n=6, five were suspected as BC and one as an upper track cancer). The inflammatory microenvironment is an essential component of all tumors, and chronic inflammation may promote BC progression. Therefore, in a patient with BC and inflammation, the patient was marked as BC. The urine samples were centrifuged for 5 minutes at 1200 rpm (in Biosan centrifuge model number: FV-2400). The samples were stored at −80 °C. Before the use of samples, samples were defrosted at room temperature.

### Electrochemical Measurements

The sample was added to a 20 ml electrochemical cell fitted with a cell cap with the six-electrode array design shown in FIG. 8 (n_{bare}=n_{chitosan+CNT}=n_{Pt}=2). The platinum black and bare electrodes were chosen using the CR criteria (see Table 4), and the electrode coated with chitosan-CNT was chosen owing to its good sensitivity towards hydrogen peroxide. Differential pulse voltammetry was performed in the urine samples across the potential range of −0.1V to +0.7V. The electrochemical signal was recorded by using MultiPalmSens4 and the MultiTrace software.

FIGS. 9 and 10 show the voltammograms recorded by DPV of the urine samples, after the application of baseline correction and electrode fusion to the original signal. The obtained signal pattern is consistent with the theory that the redox spices are steadily depleted during the oxidation. When the anodic peak is observed, the current is dictated by the delivery of additional redox species via diffusion from the bulk solution, as the diffusion layer continues to grow with the scan, which slows down mass transport to the electrode. As a result, the diffusion rate from the bulk solution to the electrode surface decreases, resulting in a decreased current as the scan continues. After the current's peak, the voltammogram is under diffusion control, whereas at the beginning it was the electrode kinetics which controlled the response. In FIGS. 9 and 10, one can also observe a shift of the signal between different electrodes and different cancer patients and that most of the signal variability is at the oxidation peaks. At the chitosan-CNT electrode another peak was observed at 0.27 [V].

The DPV generated signal was preprocessed with baseline correction method asymmetric least squares, electrode fusion, and data normalization.

Baseline correction: baseline correction with asymmetric least square was performed, as described, for example, by PHC Eilers HB. Baseline correction with asymmetric least squares smoothing. Leiden Univ Med Cent Rep. 2005; 1 (1): 5 and Baek S-J, Park A, Ahn Y-J, Choo J. Baseline correction using asymmetrically reweighted penalized least squares smoothing. Analyst. 2015; 140 (1): 250-257. doi:10.1039/c4an01061b].

Electrode fusion: this step involves the following treatment. The recorded signal yₙ is given by:

yₙ=x+vₙ (5)

where x is the original signal and vₙ is the additive noise, n represents the number of the electrode. We define \tilde{x} as the estimated original signal, given by:

$$\tilde{x} = \frac{1}{N} {\sum }_{n = 1}^{N} {y}_{n} \qquad ( 6 )$$

Then, the mean estimated signal is equal to x as given by equation (7):

$$E [ \tilde{x} ] = E [ \frac{1}{N} {\sum }_{n = 1}^{N} {y}_{n} ] = \frac{1}{N} {\sum }_{n = 1}^{N} E [ {y}_{n} ] = \frac{1}{N} {\sum }_{n = 1}^{N} E [ x + {v}_{n} ] = \frac{1}{N} {\sum }_{n = 1}^{N} \underbrace{E [ x ]}_{x} + \underbrace{E [ {v}_{n} ]}_{0} = x$$

and the variance of the estimated signal is:

$$Var ( \tilde{x} ) = E [ {( \tilde{x} - E [ \tilde{x} ] )}^{2} ] = E [ {( \frac{1}{N} {\sum }_{n = 1}^{N} {y}_{n} - x )}^{2} ] = \frac{1}{{N}^{2}} {\sum }_{n = 1}^{N} E [ {( x + {v}_{n} - x )}^{2} ] = \frac{1}{{N}^{2}} {\sum }_{n = 1}^{N} E [ {v}_{n}^{2} ] = \frac{1}{{N}^{2}} {\sum }_{n = 1}^{N} {σ}_{n}^{2} = \frac{{σ}^{2}}{N} \qquad ( 8 )$$

Therefore, variance is reduced by factor N (N=2, as a pair of electrodes of each type is used).

Data normalization: Some features comprise higher values (mean and variance) with less information about the analyte of interest. The standard scaling denoted by z is defined in equation (9):

$$Z = \frac{x - μ}{σ} \qquad ( 9 )$$

Where σ is the standard deviation and μ is the mean.

Classification between BC and non-BC cancer patients and classification between PC and non-PC cancer patients: the data was split into a train (90%) and test (10%) and validated with stratified 7-fold cross validation. Grid search was performed on the models space (ANN or Partial Least Square Discriminant Analysis (PLS-DA)) on the original data and the extracted features with the python package ‘tsfresh’, on the number of features (3, 5, 8, 13 for each electrode) that were selected according to F-value with the python package SelectKBest, in case of ANN number of nodes in the hidden layer (3, 5, 8, 13, 20, 30, 50).

ANN emerged as the best model for differentiating BC patients or PC patients from non-BC or non-PC patients. The characteristics and performance of the ANN model from the grid search are tabulated below in Table 5A-5B (BC) and 5C-5D (PC):

TABLE 5A
BC Train (90%)

| | Number of features | | Sensi- | | |
|---|---|---|---|---|---|
| model | for each electrode | Nodes | tivity | Specificity | accuracy |
| ANN | 1 | 8 | 0.85 | 0.85 | 0.85 |

TABLE 5B
BC test (10%)

| | Number of features | | Sensi- | | |
|---|---|---|---|---|---|
| model | for each electrode | Nodes | tivity | Specificity | accuracy |
| ANN | 1 | 8 | 0.88 | 0.88 | 0.88 |

TABLE 5C
PC Train (90%)

| | Number of features | | Sensi- | | |
|---|---|---|---|---|---|
| model | for each electrode | Nodes | tivity | Specificity | accuracy |
| ANN | 8 | 20 | 0.86 | 0.86 | 0.86 |

TABLE 5D
PC test (10%)

| | Number of features | | Sensi- | | |
|---|---|---|---|---|---|
| model | for each electrode | Nodes | tivity | Specificity | accuracy |
| ANN | 8 | 20 | 0.63 | 0.63 | 0.63 |

It is seen that urine samples from BC patients were classified versus all other samples with sensitivity and specificity of 0.88. Urine samples from PC patients were classified versus all other samples with sensitivity and specificity of 0.63.

Classification Between BC Patients and Patients with Inflammation:

The data was split into a train (80%) and test (20%) and validated with stratified 4-fold cross validation. Grid search was performed on the models' space (ANN or PLS-DA), on the original data and extracted features. In the case of ANN number of nodes in the hidden layer (3, 5, 8), the features were selected according to F-value with the python package SelectKBest. Moreover, the ANN models were fully connected (dense) neural network applied with one hidden layer, adaptive moment estimation optimizer, and loss function of binary cross-entropy.

The best model performance of the grid search was obtained with PLS-DA model, with one extracted feature for each electrode. The model performance is 87.5%, 83.3% accuracy, 87.5%, 83.3% sensitivity, 12.5%, 16.6% FPR, and 87.58, 83.3% precision for the train and the test, respectively.

### Example 3

Voltammetry Measurements in Urine Samples of Patients Diagnosed with Cancer Using an Array of Eight Working Electrodes and Signal Analysis with Chemometrics

The goal of the study was to differentiate between the redox fingerprints of BC patients and healthy volunteers using a commercial screen-printed electrode (SPE) with 8 surface modified carbon electrodes of 2.95 mm diameter. The working electrodes were sharing a silver reference electrode, and a carbon counter electrode.

### Urine Sample Preparation

A total of fifty-nine (59) fresh urine samples (2 ml in size) were collected from twenty-five (25) bladder cancer patients. The group consisted of eighteen (18) men and seven (7) women, with age between 49 to 86 years old. In addition, twenty-five (25) fresh urine samples (2 ml in size) were collected from thirteen (13) healthy volunteers.

### Electrochemical Measurements

A drop of 2 ml fresh urine was placed on the modified commercial SPE, as shown in FIG. 13B. 2-ways differential pulse voltammetry was performed in the urine samples across the potential range of −0.1V to +0.7V (Oxidation), and across the potential range of +0.7V to −0.1V (Reduction) with voltage step of 5 mV, pulse mode of 50 mV, pulse time of 0.1 sec, and scan rate of 0.02 V/sec. The electrochemical signal was recorded using Palmsens Emstat Pico MUX16 board.

FIG. 14 shows a voltammogram recorded by DPV of the urine samples both for oxidative and reduction.

Classification between BC patients and healthy volunteers: data was split into a train (70%) and test (30%) and validated with stratified 7-fold cross validation. Grid search was performed on the models space (ANN or Partial Least Square Discriminant Analysis (PLS-DA)) on the original data and the extracted features with the python package ‘tsfresh’, on the number of features (1, 3, 5, 8, 13 for each electrode) that were selected according to F-value with the python package SelectKBest, in case of ANN number of nodes in the hidden layer (3, 5, 8, 13, 20, 30, 50).

ANN emerged as the best model for differentiating BC patients from healthy volunteers. The characteristics and performance of the ANN model from the grid search are tabulated below in Table 6A-6B:

TABLE 6A
Train (70%)

| | Number of features | | Sensi- | | |
|---|---|---|---|---|---|
| Model | for each electrode | Nodes | tivity | Specificity | Accuracy |
| ANN | 30 | 50 | 95% | 86% | 94% |

TABLE 6B
Test (30%)

| | Number of features | | Sensi- | | |
|---|---|---|---|---|---|
| Model | for each electrode | Nodes | tivity | Specificity | Accuracy |
| ANN | 30 | 50 | 94% | 88% | 92% |

It is seen that urine samples from BC patients were classified versus healthy volunteers with sensitivity of 94% and specificity of 88%.

1) A method of screening and/or monitoring a patient for cancer that is detectable in urine, comprising:
obtaining a urine sample from the patient;
acquiring an electrochemical signal generated jointly by redox molecules in the urine sample, wherein the signal is acquired with the aid of an array comprising surface-modified electrodes that is sensitive for antioxidant and oxidant, wherein the array optionally further comprises one or more bare electrodes; optionally preprocessing the electrochemical signal, to obtain processed data;
applying trained chemometric model(s) to the processed or raw data; and
determining the presence of cancer and/or monitorable cancer-related features, after the chemometric model has classified the processed data according to its training data set.

2) The method according to claim 1, wherein the cancer is urologic cancer.

3) The method according to claim 1, wherein the electrochemical signal is acquired by voltammetry.

4) The method according to claim 1, wherein the urine sample is placed in electrochemical measurement cell equipped with a counter electrode, optionally a reference electrode and an array of working electrodes that comprises at least one working electrode showing sensitivity above 3.0 mA/M for ascorbic acid and at least one working electrode showing sensitivity above 3.0 mA/M for hydrogen peroxide.

5) The method according to claim 4, wherein the array comprises at least one working electrode showing partial selectivity for ascorbic acid/hydrogen peroxide of above 5 and at least one working electrode showing partial selectivity for hydrogen peroxide/ascorbic acid of above 2.

6) The method according to claim 4, wherein the array of working electrodes comprises at least two sets of electrodes selected from:
1) a set consisting of one or more bare electrodes;
2) a set of consisting of one or more electrodes coated with polysaccharide film;
3) a set consisting of one or more electrodes coated with polysaccharide film with conductive additives incorporated into the film;
4) a set consisting of one or more electrodes coated with reduced graphene oxide film;
5) a set consisting of one or more electrodes coated with platinum black film;
6) a set consisting of one or more electrodes coated with MoS₂^{A} film;
7) a set consisting of one or more electrodes coated with MoS₂^{B} film;
8) a set consisting of one or more electrodes coated with WS₂^{A} film; and
9) a set consisting of one or more electrodes coated with WS₂^{B} film;
wherein the superscripts A and B indicate electrodeposited MoS₂ and WS₂ coatings produced by cycling across potential ranges A and B, respectively, wherein potential range A corresponds to the double layer potential region of the working electrode and potential range B extends to more positive potentials than potential range A.

7) The method according to claim 6, wherein the array of working electrodes comprises a set consisting of 1 to 3 bare electrodes and a set consisting of 1 to 3 platinum black coated electrodes.

8) The method according to claim 7, wherein the array of working electrodes further comprises a set consisting of from 1 to 3 electrodes coated with polysaccharide film with conductive additives incorporated into the film.

9) The method according to claim 6, wherein the signal is acquired with the aid of an array of electrodes placed in an annular space between a ring-shaped counter electrode and a centrically positioned reference electrode.

10) The method according to claim 6- or 9, wherein the array of working electrodes comprises from two to five of the following types of electrodes:
bare electrode, chitosan/CNT-coated electrode, platinum black-coated electrode, reduced graphene oxide-coated electrode, MoS2^{A}-coated electrode, MoS2^{B}-coated electrode, WS2^{A}-coated electrode, and WS2^{B}-coated electrode.

11) The method according to claim 6, wherein the array of working electrodes comprises from six to eight of the following types of electrodes:
bare electrode, chitosan/CNT-coated electrode, platinum black-coated electrode, reduced graphene oxide-coated electrode, MoS2^{A}-coated electrode, MoS2^{B}-coated electrode, WS2^{A}-coated electrode, and WS2^{B}-coated electrode.

12) The method according to claim 6, wherein the array of working electrodes comprises at least one bare electrode, at least one chitosan/CNT-coated electrode, at least one platinum black-coated electrode, at least one reduced graphene oxide-coated electrode, at least one electrode, at least one coated electrode, at least one electrode and at least one WS2^{B}-coated electrode.

13) The method according to claim 1, wherein the preprocessing the electrochemical signal comprises one or more of baseline correction, noise reduction, electrode fusion and data normalization.

14) The method according to claim 1, wherein the chemometric model applied is an artificial neural network-based model (ANN) or Partial Least Square Discriminant Analysis (PLS-DA).

15) The method according to claim 14, wherein during preprocessing, from 1 to 50 features are extracted for each electrode.

16) The method according to claim 14, wherein from 1 and 50 nodes are applied in the ANN.

17) The method of monitoring a patient for cancer according to claim 1, comprising determining one or more of: recurrence and/or disease progression, tumor classification, and treatment response.

18) An electrochemical sensor consisting of:
one or more bare electrodes;
one or more polysaccharide-coated electrodes with conductive additives incorporated into the coating; and
one or more platinum black-coated electrodes.

19) A device for electrochemical detection, comprising:
an electrochemical sensor as defined in claim 18;
a counter electrode and optionally a reference electrode;
a potentiostat or galvanostat to which the working electrodes, the counter electrode and optionally the reference electrodes are electrically connected to allow control of the potential or current of the working electrodes, respectively, to create an electrochemical signal when the electrodes are immersed in a sample; and
a processor configured to analyze the electrochemical signal by one or more chemometric techniques.
